SEQUENCE LISTING (10 sequences)

SEQ ID NO: 1 - protein, 20 aa, Artificial sequence
  Synthetic Peptide
AKLTALTLLGLGLALFDGQK

SEQ ID NO: 2 - protein, 7 aa, Artificial sequence
  Synthetic Peptide
IFYYDPK

SEQ ID NO: 3 - protein, 11 aa, Artificial sequence
  Synthetic Peptide
ATNDHYFADPY

SEQ ID NO: 4 - protein, 20 aa, Artificial sequence
  Synthetic Peptide
AKLTALTELGLGLALFDGQK

SEQ ID NO: 5 - protein, 7 aa, Artificial sequence
  Synthetic Peptide
IFEYDPK

SEQ ID NO: 6 - protein, 11 aa, Artificial sequence
  Synthetic Peptide
ATNDHEFADPY

SEQ ID NO: 7 - protein, 20 aa, Artificial sequence
  Synthetic Peptide
AKLTALTKLGLGLALFDGQK

SEQ ID NO: 8 - protein, 7 aa, Artificial sequence
  Synthetic Peptide
IFKYDPK

SEQ ID NO: 9 - protein, 11 aa, Artificial sequence
  Synthetic Peptide
ATNDHKFADPY

SEQ ID NO: 10 - protein, 11 aa, Artificial sequence
  Synthetic Peptide
THLAPYSDELR

(19) United States
(12) Patent Application Publication
(10) Pub. No.: US 2017/0115280 A1
(43) Pub. Date: Apr. 27, 2017

(54) AFFINITY PROBES FOR DEFINING PROTEIN-LIPID CONTACTS

(71) Applicant: The Cleveland Clinic Foundation, Cleveland, OH (US)

(72) Inventors: Stanley L. Hazen, Pepper Pike, OH (US); Xiaodong Gu, Cleveland, OH (US)

(21) Appl. No.: 15/333,871

(22) Filed: Oct. 25, 2016

Related U.S. Application Data
(60) Provisional application No. 62/246,297, filed on Oct. 26, 2015.

(51) Int. Cl. G01N 33/53; G01N 33/92

(52) U.S. Cl. CPC G01N 33/5306; G01N 33/92

(57) ABSTRACT

Provided herein are activatable and taggable lipid probes, and method of use thereof. In particular, the probes described herein find use in the identification of protein-lipid interactions.

The present application claims priority to U.S. Provisional application Ser. No. 62/246,297, filed Oct. 26, 2015, which is herein incorporated by reference in its entirety.

### FIELD

Provided herein are activatable and taggable lipid probes, and method of use thereof. In particular, the probes described herein find use in the identification of protein-lipid interactions.

### BACKGROUND

Since the Framingham heart study revealed the inverse relationship between high-density lipoprotein (HDL) cholesterol levels and the risk of coronary artery disease (CAD), the structure and biological functions of HDL have been under intense scrutiny (refs. 1-6; herein incorporated by reference in their entireties). HDL and its major structural protein component, apolipoprotein A-I (apoA-I), play a pivotal role in mediating reverse cholesterol transport (RCT), which describes the process of cholesterol removal from peripheral tissues for ultimate elimination from the body by excretion (refs. 7-10; herein incorporated by reference in their entireties). Although facilitation of RCT through cholesterol acceptor activity is considered a major function of HDL particles, ample evidence shows additional functions exist, such as anti-oxidant, anti-inflammatory and anti-tumorigenic activities, and the shuttling of microRNAs or lipid signaling molecules (refs. 11-17; herein incorporated by reference in their entireties). HDL as a highly heterogeneous array of distinct particles, with differing associated proteome and peptidome (refs. 18-23; herein incorporated by reference in their entireties). In addition to exchangeable apolipoproteins, early proteomics studies revealed the HDL associated proteome includes known participants critical to hemostasis and thrombosis, innate and adaptive immune function, growth factors and various receptors, and hormone-associated proteins (refs. 18,21). In the years that have followed since the original HDL proteomics studies, subsequent reports have confirmed and expanded upon these earlier findings, revealing a remarkably complex HDL associated proteome that can differ as a function of disease status or susceptibility (refs. 23-26). Thus, alterations in HDL function appear to be, at least in part, dictated by changes in the compliment of HDL-associated proteins present, with distinct HDL particle subpopulations composed of unique and dynamically changing clusters of specific HDL-associated proteins (refs. 25-27). Improved tools are needed for defining proteins and protein residues involved in interactions with membranes and lipoprotein surfaces.

### SUMMARY

Provided herein are activatable and taggable lipid probes, and method of use thereof. In particular, the probes described herein find use in the identification of protein-lipid interactions.

In some embodiments, provided herein are compositions comprising: (a) a lipid moiety, (b) an activatable moiety, and (c) a taggable moiety; wherein the activatable moiety and the taggable moiety are independently covalently linked to the lipid moiety.

In some embodiments, the activatable moiety is a photoactivatable moiety. In some embodiments, the photoactivatable moiety comprises a functional group selected from the group consisting of: an aryl azide, an azido-methyl-coumarin, a benzophenone, an anthraquinone, a diazo compound, a diazirine ring, and a psoralen derivative. In some embodiments, the photoactivatable moiety comprises a diazirine ring.

In some embodiments, the taggable moiety is a clickable moiety. In some embodiments, the clickable moiety comprises a function group selected from the group consisting of: an alkyne, an azide, a transcyclooctene, a tetrazine groups, and a dibenzocyclooctyne. In some embodiments, the clickable moiety comprises an alkyne. In some embodiments, the alkyne is a terminal alkyne.

In some embodiments, the lipid moiety is selected from the group consisting of: a phospholipid, a glyceride, a sphingolipid, an eicosanoid, and a fatty acid. In some embodiments, the lipid moiety is a glyceride selected from a monoglyceride, diglyceride, and triglyceride. In some embodiments, the lipid moiety is a phospholipid. In some embodiments, the phospholipid is selected from phosphatidylethanolamine (PE), phosphatidylcholine (PC), phosphatidylserine (PS), phosphatidylglycerol (PG), phosphatidylinositol (PI), glycophosphatidylinositol (GPI), plasmalogens, cardiolipin, and sphingomyelin.

In some embodiments, provided herein are lipid-based probes comprising a general structure depicted in one of FIGS. 13-21, wherein: A is a activatable moiety (e.g., photoactivatable moiety (e.g., diazirine ring)), T is a taggable moiety (e.g., clickable moiety (e.g., alkyne)), and wherein X is a head group selected from choline, inositol, ethanolamine, serine, or other standard or non-standard lipid head groups.

In some embodiments, provided herein are methods of preparing a lipid-based probe comprising appending or inserting one activatable group and one taggable group onto or within the fatty acid chains and/or head group of a lipid molecule.

In some embodiments, provided herein are systems comprising a lipid-based probe described herein and a tagging element comprising a functional portion and a reactive portion, wherein the reactive portion has the capacity to interact with the taggable moiety of the lipid-based probe to form a covalent bond or stable non-covalent interaction. In some embodiments, the taggable moiety and reactive portion of the tagging agent are selected from: alkyne and azide groups, transcyclooctene and tetrazine groups, and dibenzocyclooctyne and azide groups.

In some embodiments, provided herein are methods of associating a functional portion of a tagging agent with a protein or protein complex within a lipid-containing structure, comprising: (a) contacting the lipid-containing structure or a system comprising the lipid-containing structure with a lipid-based probe described herein, such that the lipid-based probe is incorporated into the lipid-containing structure; (b) activating the activatable moiety to initiate formation of covalent bonding with the protein or protein complex; and (c) contacting the taggable moiety of the lipid-based probe with the tagging agent, such that a reactive portion of the tagging agent forms a covalaent bond or stable non-covalent interaction with the taggable moiety, thereby associating the functional portion of the tagging agent with the protein or protein complex within the lipid-containing structure via the lipid-based probe.

In some embodiments, provided herein are methods of identifying interactions between a protein or protein complex and lipids within a lipid-containing structure, comprising: (a) contacting the lipid-containing structure or a system comprising the lipid-containing structure with a lipid-based probe described herein, such that the lipid-based probe is incorporated into the lipid-containing structure; (b) activating the activatable moiety to initiate formation of covalent bonding with the protein or protein complex; (c) contacting the taggable moiety of the lipid-based probe with the tagging agent, such that a reactive portion of the tagging agent forms a covalaent bond or stable non-covalent interaction with the taggable moiety; (d) isolating the protein or protein complex using a functional portion of the tagging agent; and (e) analyzing the protein or protein complex to identify residues bound to the lipid-based probe.

### BRIEF DESCRIPTION OF THE DRAWINGS

FIG. 1. Design and chemical synthesis of the lipid probe pac-PC. (A) Chemical structure of pac-PC. Pac-PC is an analogue of phosphatidylcholine, with two extra function groups: one diazirine group (in dashed circle) on sn-3 phosphate terminal and one alkyne group (in dashed square) on sn-2 fatty acid side chain terminal. The carbene radical (in dashed circle) is generated after UV irradiation. Terminal alkyne reacts with biotin azide to give a triazole (in dashed square). (B) The chemical synthesis of pac-PC began from a commercially available lipid (1,2-dimyristoyl-sn-glycero-3-phosphate, DMPA). After being coupled with 2-(3-methyl-3H-diazirin-3-yl)ethanol, the sn-2 side chain was removed through phospholipase A₂ (PLA2) catalyzed hydrolysis. Further reaction of the lyso-lipid with 10-undecynoic acid in the presence of coupling reagents dicyclohexylcarbodiimide (DCC) and 4-dimethylaminopyridine (DMAP) delivered the final product, pac-PC.

FIG. 2. Experimental scheme of using pac-PC to investigate pac-PC/apoA-I interaction. Reconstituted nascent HDL (rHDL) was prepared and contained 2 mole % of pac-PC. After UV irradiation, the pac-PC/apoA-I cross-linking products were biotinylated using click chemistry. Biotin tagged pac-PC/apoA-I adducts were enriched by Neutravidin collection, trypsinized, and unbound peptides washed away. Following the wash step, pac-PC modified apoA-I peptides were eluted from the beads. In other experiments, base hydrolysis of pac-PC modified peptides was incorporated to remove the bulk of the lipid side chains allowing accurate software interrogation of modified peptide adducts by analysis using LC/MS/MS.

FIG. 3. Peptides labeled with pac-PC have unique fragmentation patterns. (A) One representative tandem mass spectrum of pac-PC modified apoA-I peptide (Thr₁₆₁-Arg₁₇₁) having the diagnostic fragments m/z 284 and m/z 775. (B) Tandem mass spectrum of pac-PC after photoactivation (in PBS buffer) and click chemistry reaction, which has dominant fragments m/z 284 and m/z 775.

FIG. 4. A base hydrolysis step effectively removes the bulk of the lipid side delivering structurally informative and recognizable peptide backbone fragmentations. Tandem mass spectrum of pac-PC modified apoA-I peptide (Thr₁₆₁-Arg₁₇₁) (SEQ ID NO: 10) after base hydrolysis. Amino acid and b/y ions that carry hydrolyzed pac-PC modification group (+226.061) are labeled with *.

FIG. 5. Experimental scheme of using pac-PC to investigate pac-PC/rPON1 interactions. Reconstituted nascent HDL (rHDL) incorporating the 2 mole % pac-PC was incubated with His tagged rPON1 and cross-linking was performed with brief exposure to UV (365 nm) and enriched by using Ni-NTA resin. Immobilized PON1 containing phospholipid adducts were biotinylated via click chemistry and purified using Neutravidin, digested with trypsin, and processed as described in FIG. 2. Lipid adducted modified rPON1 peptides were eluted from the beads and base treated to remove bulk lipid and allow facile identification of the modification sites on rPON1 by LC/MS/MS and searching for glycerol- phosphate modified peptides, which were detected by a static modification (+226.061) applied to each amino acid residue.

FIG. 6. The illustration of tandem mass spectra from rPON1 peptide Ile₂₉₁-Lys₂₉₇ (SEQ ID NO: 2) and its cross-linking product with pac-PC. The tandem mass spectra shown are (A) rPON1 peptide Ile₂₉₁-Lys₂₉₇ (SEQ ID NO: 2) and (B) rPON1 peptide Ile₂₉₁-Lys₂₉₇ (SEQ ID NO: 2) carrying a modification (+226.061) at Tyr293. The pac-PC modified amino acid and related b, y ions are labeled with *.

FIG. 7. Crystal structure of rPON1 with highlighted residues that cross-linked with photoactivatable lipid probes. Leu9 (which is not shown on the structure), Tyr185 and Tyr293 were identified that cross-linked with pac-PC. Tyr294 was later identified that also cross-linked with pac-PC but with much lower abundance (less than 5%) compared to Tyr293, using the selective ion monitoring mode. Tyr71 was previously identified in crosslinking experiments. All of these residues are located in the contemplated HDL binding domain (above the dash line, according to PON1 crystal structure and hydropathy analyses).

FIG. 8. Significant differences in cross-linking formation between pac-PC/Tyr293 versus pac-PC/Tyr294 demonstrate the spatial sensitivity and specificity of using pac-PC to interrogate lipid-protein interactions at the interface of PON1 and HDL. (A) Chromatogram of the selective ion monitoring m/z 586.3 corresponding to rPON1 peptide Ile₂₉₁-Lys₂₉₇ carrying hydrolyzed pac-PC modification group (+226.061). A new peptide that has the same m/z value but slightly different retention time was isolated as the small shoulder peak (peak 1) on the chromatogram. (B) New isolated tandem mass spectrum of rPON1 peptide Ile₂₉₁-Lys₂₉₇ (SEQ ID NO: 2) carrying hydrolyzed pac-PC modification group (+226.061) at Tyr294.

FIG. 9. Characterizations of site-specific rPON1 mutants. All mutants were compared with the recombinant PON1 variant G3C9 (wild type like activity, rPON1 WT). Residues (Leu9, Tyr185 and Tyr293) were mutated to either glutamic acid (rPON1 pan E) or lysine (rPON1 pan K). (A) Purified rPON1 WT, pan E and pan K proteins were fractionated by 12% SDS-PAGE, and stained with Coomassie Blue. (B) Circular dichroism spectra of purified rPON1 WT, pan E and pan K proteins. rPON1 forms (WT, pan E and pan K mutants) were analyzed at ambient temperature in continuous scan mode with a 1-nm bandwidth (100000 counts/step).

FIG. 10. (A) paraoxonase activity, (B) arylesterase activity of purified rPON1 WT, pan E and pan K mutants. Purified rPON1 pan K mutant demonstrated a specific activity of ˜50% paraoxonase activity, and ˜80% arylesterase activity compared with rPON1 WT, whereas rPON1 pan E mutant demonstrated almost complete loss of activity with either substrate. Data shown represent the mean±SD of triplicate determinations.

FIG. 11. Surface plasmon resonance sensorgrams of the binding of various concentrations of rPON1. (A) WT and (B) pan K mutant to rHDL particles. rHDL was directly immobilized on a CMS sensor chip. Samples of rPON1 ranging from 500 nM to 2000 nM were prepared in PON1 activity buffers and flowed over the surface of the sensor chip. The apparent dissociation constants (K_{d}) were obtained by fitting background-subtracted SPR binding data to the 1:1 binding with drifting baseline model within the BIA evaluation software version 4.0.

FIG. 12. Kinetics of the inactivation of rPON1 (A) WT (B) pan K in the solution of excess rHDL particles, 0.1% tergitol, or buffer only. Recombinant PON1WT and pan K were pre-incubated with excess rHDLs (excess), 0.1% tergitol (NP-10), or PON1 activity buffer. Inactivation was initiated by adding an equal volume of denaturing and incubating the. Aliquots were taken at the indicated time points and arylesterase activities were examined. Data shown represent the mean±SD of triplicate determinations.

FIG. 13. General structures of exemplary phospoholipid probes: X represents a phospholipid head group such as choline, serine, inositol, ethanolamine, or a non-standard phospholipid head group; A is an activatable group; T is a taggable moiety; and n1, n2, and n3 are independently 0-28.

FIG. 14. General structures of exemplary phospoholipid probes having a phospholipid head group at the sn-3 position: X represents a phospholipid head group such as choline, serine, inositol, ethanolamine, or a non-standard phospholipid head group; A is an activatable group; T is a taggable moiety; and nl, n2, n3, and n4 are independently 0-28.

FIG. 15. General structures of exemplary phospoholipid probes having a diazirine ring photoactivatable moiety, and a terminal alkyne clickable moiety: X is absent or is a phospholipid head group such as choline, serine, inositol, ethanolamine, or a non-standard phospholipid head group; and n1, n2, n3, n4, and when present n5 are independently 0-28.

FIG. 16. Structures of exemplary phospoholipid probes having one or more diazirine ring photoactivatable moieties and one or more terminal alkyne clickable moieties; n1, n2, n3, and n4 (when present) are independently 0-28.

FIG. 17. Structures of exemplary phosphatidylcholine probes having one or more diazirine ring photoactivatable moieties and one or more terminal alkyne clickable moieties; n1, n2, n3, and n4 (when present) are independently 0-28.

FIG. 18. General structures of exemplary triglyceride probes: A is an activatable group; T is a taggable moiety; and n1, n2, n3, n4, and n5 are independently 0-28.

FIG. 19. Structures of exemplary triglyceride probes one or more diazirine ring photoactivatable moieties and one or more terminal alkyne clickable moieties; n1, n2, n3, n4 (when present), and n5 (when present) are independently 0-28.

FIG. 20. General structures of exemplary diglyceride probes: A is an activatable group; T is a taggable moiety; and n1, n2, n3, n4, and n5 are independently 0-28.

FIG. 21. Structures of exemplary diglyceride probes having a diazirine ring photoactivatable moiety and a terminal alkyne clickable moiety; n1, n2, n3, and n4 are independently 0-28.

FIG. 22. Structures of exemplary photoactivatable moieties.

FIG. 23. Major MS/MS fragments of pac-PC modified peptides come from lipid portion. The control experiment was performed by using pac-PC vesicles in PBS buffer. For comparison, the steps for use and analysis of pac-PC coupled peptides following interaction with protein (as in rHDL) are also shown. After UV irradiation, pac-PC is biotinylated through click chemistry and affinity isolated, subjected to trypsinolysis, and analyzed by LC/MS/MS. In the example of only pac-PC exposed to UV irradiation in buffer alone, the final extracted lipid gave m/z 284 and m/z 775 as major MS/MS fragments, typical patterns from pac-PC modified peptides. Thus, fragment ions m/z 284 and m/z 775 originate from the lipid portion of lipid-adducted peptides.

FIG. 24. PON1 adjacent residues Tyr293 and Tyr294 play different roles in PON1 activity and stabilization of PON1 through HDL interaction. (A) The arylestrase activity of rPON1 WT, Y293K, and Y294K mutants in the presence of rHDL are shown. (B) Kinetics of the inactivation of rPON1 WT, Y293K, and Y294K mutants in the presence of excess HDL particles is shown. rPON1WT, Y293K, and Y294K were pre-incubated with rHDLs, and inactivation was initiated by adding an equal volume of denaturing buffer (containing metal chelator and reducing agent) and incubating the samples at 37° C. Aliquots were taken at the indicated time points and arylesterase activity in reactions was determined. Data shown represent the mean±SD of triplicate determinations.

### DEFINITIONS

As used herein, the term “activatable” refers to a compound or moiety that has a latent capacity or feature which can be made active by exposure to a specific condition or stimulus. For example, a functional group on a probe is non-reactive in its latent state, but is rendered reactive (e.g., capable of crosslinking to protein) by exposure to a stimuli (e.g., light, heat, an activator molecule, etc.). Such a group may be termed a “activatable crosslinking moiety.” The term “photoactivatable” refers to an activatable compound or moiety that is activated by exposure to light (e.g., at a particular wavelength range (e.g., UV light)).

As used herein, the term “taggable” refers to a compound or moiety that forms a covalent bond or stable noncovalent bond when contacted or in proximity of a “tagging” compound or agent. The “tagging” compound or moiety comprises a reactive portion which forms the bond with the taggable compound or moiety and a functional portion which facilitates detection or isolation of a tagged compound or complex. The term “clickable” refers to a compound or moiety displaying a functional group that enables tagging with a “clicking” compound or moiety via click chemistry. Suitable clickable/clicking pairs include azide/alkyne and thiol/alkene.

### DETAILED DESCRIPTION

Provided herein are photoactivatable and taggable (e.g., clickable) lipid (e.g., phospholipid) probes that are useful to, for example, interrogate protein contact residues that support interactions (e.g., tight binding interactions) with lipids. In some embodiments, probes are synthetic photoactivatable and taggable phospholipid (e.g., phosphatidylcholine) analogues (e.g., photoactivatable and clickable phospholipid (pac-PL), such as photoactivatable and clickable phosphatidylcholine (pac-PC)).

Using paraoxonase 1(PON1)-HDL interactions as a test case (See, e.g., Examples 1 and 2), experiments were conducted during development of embodiments of the present invention to illustrate the facile use of the photoactivatable probes (e.g., pac-PC) in defining key contacts between proteins and lipid-containing structures. The exemplary pac-PC probe was used to identify residues on PON1 essential for PON1 docking to HDL, PON1 catalytic activity, and stability. The pac-PC probe and other photoactivatable phospholipid probes incorporate into lipoprotein particles (e.g., HDL) or membrane bilayers and provide a strategy to facilitate identification of residues critical for protein docking with the lipid surface of lipoproteins or cell membranes.

PON1 is a high-density lipoprotein (HDL)-associated protein with atherosclerosis-protective and systemic anti-oxidant functions. It has been demonstrated that PON1, myeloperoxidase (MPO), and HDL bind to one another in vivo forming a functional ternary complex (Huang, Y. et al J. Clin. Invest. 2013 123(9):3815-28; herein incorporated by reference in its entirety). However, the specific residues on PON1 involved in the HDL-PON1 interaction were unknown prior to the experiments conducted during development of embodiments herein. Unambiguous identification of protein residues involved in docking interactions to lipid surfaces posed considerable methodological challenges in the absence of the compositions and methods described herein. However, the synthetic photoactivatable and taggable (e.g., by click chemistry) phospholipid probes described herein, when incorporated into HDL, were used to identify amino acid residues on PON1 that directly interact with the lipoprotein phospholipid surface. Several specific PON1 residues (Leu9, Tyr185 and Tyr293) were identified through covalent cross-links with the lipid probes using affinity isolation coupled to liquid chromatography with on-line tandem mass spectrometry. Based upon the crystal structure for PON1, the identified residues are all localized in relatively close proximity on the surface of PON1, defining a domain that binds to the HDL lipid surface. Site-specific mutagenesis of the identified PON1 residues (Leu9, Tyr185 and Tyr293), coupled with functional studies, reveals their importance in PON1 binding to HDL, and both PON1 catalytic activity and stability. Specifically, the residues identified on PON1 provide important structural insights into PON1-HDL interaction. Beyond the PON1 example described herein, the photoactivatable and affinity tagged lipid probes described herein provide a generalizable and valuable tool for identifying contact sites supporting protein interactions with lipid interfaces, such as on cell membranes or lipoproteins.

The above use of an exemplary probe (further detailed in Examples 1 and 2) provides important structural insights into PON1-HDL interaction. However, this is just one example of the utility of the pac-PC probe specifically, and pac-PL and pac-L probes more generally.

Plasma HDL particles represent an ensemble of lipidated apoA-I core lipoprotein (75% protein mass of HDL) with numerous distinct interacting HDL-associated proteins. Despite the interest in HDL biology and role in cardiovascular disease, an understanding of the structural regions on virtually all HDL-associated proteins critical for docking to the lipoprotein lipid surface is poorly defined. This is because few specific and effective tools exist for direct detection of protein contact points with lipid surfaces (e.g., lipoprotein, a cell membrane, microparticle, etc.). The probes described herein address this unmet need.

The activatable (e.g., photoactivatable) and taggable (e.g., clickable) lipid probes described herein provide valuable tools for identifying contact sites supporting protein interactions with lipid interfaces such as found on cell membranes or lipoproteins.

The present disclosure is not limited to the pac-PC probe exemplified with PON1 in Example 1 and 2. In some embodiments, activatable and taggable lipid probes are provided based on other phospholipids (e.g., phosphatidic acid (PA), phosphatidylethanolamine (PE), phosphatidylcholine (PC), phosphatidylserine (PS), phosphatidylinositol (PI), phosphatidylinositol phosphate (PIP), phosphatidylinositol bisphosphate (PIP2), phosphatidylinositol triphosphate (PIP3), ceramide, sphingophospholipid, etc.). Similarly, activatable and taggable lipid probes are provided based on non-phospholipid lipids, such as fatty acids, glycerolipids (e.g., monoglycerides, diglycerides, triglycerides, etc.), sphingolipids, sterol lipids (e.g., cholesterol, estrogens, androgens, progestogens, etc.), etc. Other alterations of lipids or lipid-based probes include alterations in the chain length of fatty acids (e.g., at the sn-1, sn-2, and/or sn-3 positions), switching of the fatty acid at sn-1 and alkyne at sn-2 position of the lipid (e.g., swapping the positon of the alkyne or other taggable moiety).

In some embodiments, the lipid moieties of the probes described herein comprise 1-3 fatty acid chains. A lipid may comprise a short chain fatty acid (carbon chain of <6 carbons (e.g., formic acid, acetic acid, propionic acid, butyric acid, isobutyric acid, valeric acid, isovaleric acid, etc.)), a medium chain fatty acid (carbon chain of 6-12 carbons (e.g., caproic acid, caprylic acid, capric acid, lauric acid, etc.)), a long chain fatty acid (carbon chain of 13-21 carbons (e.g., myristic acid, palmitic acid, stearic acid, arachidic acid, etc.)), a very long chain fatty acid (carbon chain of >21 carbons (e.g., behenic acid, lignoceric acid, cerotic acid, etc.)), and/or any suitable combinations thereof.

In some embodiments, the lipid moieties of the probes described herein comprise fatty acids which may be saturated or unsaturated. Unsaturated fatty acids have one or more double bonds between carbon atoms. Unsaturated fatty acids in the lipid moieties herein may be cis or trans. Examples of unsaturated fatty acids that may find use in some embodiments include, but are not limited to Myristoleic acid, Palmitoleic acid, Sapienic acid, oleic acid, elaidoc acid, vaccenic acid, linoleic acid, linoelaidic acid, α-linolenic acid, arachidonic acid, eicosapentaenoic acid, erucic acid, docosahexaenoic acid, etc.

In some embodiments, probes described herein comprise a lipid moiety displaying a activatable (e.g., photoactivatable (e.g., diazirine ring, etc.)) moiety and a taggable (e.g., clickable (e.g., terminal alkyne (CCH), etc.)) moiety. As addressed above, in some embodiments, probes are not limited by the type of lipid or the location of attachment of the activatable and taggable moieties. Additionally, in some embodiments, probes are not limited by the identity of the activatable and taggable moieties they comprise.

The probes herein comprise an activatable moiety. The activatable moieties herein exist in a first state (e.g., inert state, latent states, nonreactive states, etc.) which is nonreactive, or of very low reactivity (e.g., with proteins or other molecules present in in vivo or in vitro biological systems). However, upon activation by a triggering event or agent, the activatable moiety is converted into a second, reactive state that allows the activatable moiety to form covalent crosslinks with nearby molecules, macromolecules, and/or complexes (e.g., proteins or protein complexes). Depending upon the activatable moiety selected for a probe or particular application, different ranges and durations of reactivity are achievable. Longer durations or reactivity result in the identification of ‘looser’ and more transient interactions with the probe; whereas, short durations or reactivity result in the identification of ‘tight’ or slow-exchanging interactions with the probe.

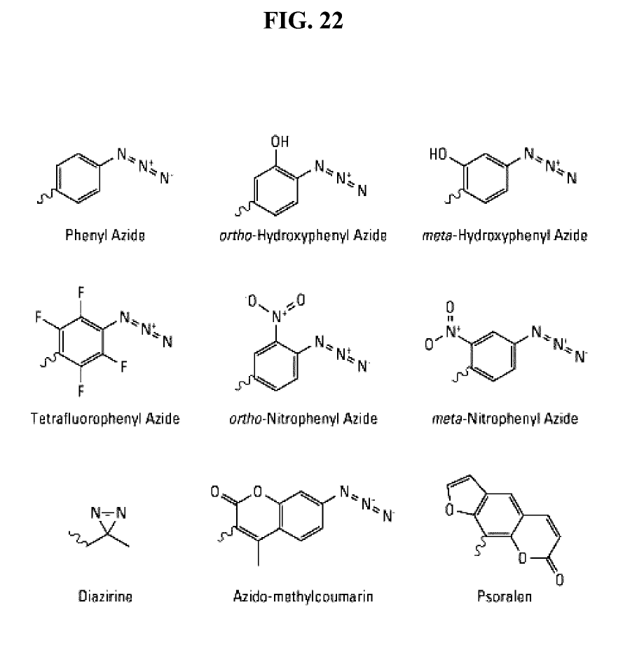

In some embodiments, an activatable moiety is a photoactivatable moiety. In such embodiments, exposure of the moiety to a particular range of wavelengths of light results in conversion of the activatable group from its non-reactive (or low reactivity) state to the reactive (crosslinking) state. The particular range of wavelengths will be selected based upon the identity of the activatable moiety. Wavelengths from about 100 to about 800 nm (e.g., 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, or any suitable ranges there between) may find use in some embodiments. Photoactivatable moieties that may find use in certain embodiments herein include, but are not limited to aryl azides, azido-methyl- coumarins, benzophenones, anthraquinones, diazo compounds, diazirines, and psoralen derivatives. Structures of exemplary photoactivatable moieties are depicted in FIG. 22.

In some embodiments, the activatable moiety is a diazirine ring. Diazirine groups are chemically stable and substantially non-reactive functional groups, even in a reducing or oxidizing environment. However, upon irradiation with UV light (e.g., wavelengths ranging from 300 to 450 nm (e.g., 300 nm, 320 nm, 340 nm, 360 nm, 380 nm, 400 nm, 420 nm, 440 nm, and any suitable ranges therein)) diazirine ring rapidly lyse to yield a highly reactive diradical carbene moiety. Due to their high reactivity the diradicals will be rapidly scavenged by water, with a near diffusion-limited rate. However, if, prior to scavenging of the radicals, the carbene is in the immediate vicinity of, for example, a C—H, C—C, C±C, N—H, O—H, S—H, etc., the carbene will inset into the bond, thereby covalently linking a compound or complex attached to the carbene to a compound of complex attached to the bond. As a result of this rapid reactivity, irradiation of a diazirine ring, and formation of a diradical carbene, will result in rapidly insertion into a proximate protein back bones or side chains in nanoseconds. A probe containing the diazirine ring, will thereby become crosslinked to a protein in close contact with the diazirine ring at the time of irradiation. Because of its capacity to rapidly convert from a latent or inert, non-reactive state (the diazirine structure) to a highly reactive, covalent-bond-forming state (the diradical carbene), a diazirine ring is an attractive activatable moiety for use in the lipid probes described herein. Further, because the diradical carbene is so rapidly scavenged by water, rendering it unreactive once again, crosslinks are only formed with portions of proteins that are in the immediate vicinity of the diazirine ring at the time of irradiation.

The probes herein comprise a taggable moiety. In some embodiments, a taggable moiety comprises a functional group on a probe that is capable of forming a covalent bond or stable noncovalent interaction with a tagging agent. In some embodiments, a tagging agent comprises a reactive portion (e.g., a portion that forms a stable interaction with a taggable moiety) and a functional portion (e.g., a portion that provides functionality to the tagged probe). In some embodiments, appropriate taggable moieties and tagging agents (e.g., reactive portions of tagging agents) are selected as a pair for use in embodiments herein. In some embodiments, when a tagging agent is added to a system containing a lipid probe comprising a taggable moiety, a stable interaction (e.g., covalent bond, stable noncovalent interaction) is formed between the reactive portion of the tagging agent and the taggable moiety. In some embodiments, the formation of this stable interaction results in the association of the functional portion of the tagging agent with the lipid probe. Depending upon the identity of the functional portion of the tagging agent, tagging of the lipid probe provides for isolation, detection, localization, characterization, etc. of the lipid probe, any proteins or complexes associated with the activatable moiety of the probe, etc.

Suitable taggable moiety/tagging agent (e.g., reactive portion) pairs are functional groups or compounds that rapidly and reliably form stable interactions under biological-like conditions. Exemplary pairs are alkyne and azide, thiol and maleimide, thiol and haloacetyl (e.g., iodoacetyl, etc.), thiol and pyridyl disulfide (e.g. pyridyldithiol, etc.), sulphonyl azides and thio acids, etc. In some embodiments, either member of the pair may find use as the taggable moiety or the tagging agent. Other functional-group pairs that form covalent bonds or other stable interactions may also be used.

In some embodiments, a taggable moiety is a clickable moiety, capable of rapid covalent bonding with a clicking agent (e.g., reactive portion or a clicking agent). Exemplary pairs of clickable moieties and clicking agents include, but are not limited to alkyne and azide groups, transcyclooctene and tetrazine groups, dibenzocyclooctyne and azide groups, etc. In some embodiments, either member of the pair may find use as the clickable moiety or the clickable agent. Other functional-group pairs capable of click chemistry may also be used.

In some embodiments, the functional portion of a tagging agent is a compound, functional group, or other agent that is covalently bound to the reactive portion of the tagging agent and provides the tagging agent with a useful functionality. In some embodiments, upon reaction of the reactive portion of the tagging agent with the taggable moiety, the functionality of the functional portion of the tagging agent is imparted onto the probe (and any proteins or complexes with which the activatable moiety and/or lipid moiety of the probe are stably associated). Attachment of the tagging agent to the probe allows for use of the functional portion of the tagging agent to isolate, detect, characterize, localize, quantitate, etc. the probe and any proteins or complexes with which it is associated.

Exemplary functional portions of tagging agents for use in the embodiments herein include, but are not limited to: amino acids (e.g., a naturally occurring amino acid or a non- natural amino acid), a peptide or polypeptide (protein) including an antibody or a fragment thereof, a His-tag, a FLAG tag, a Strep-tag, an enzyme, a cofactor, a coenzyme, a peptide or protein substrate for an enzyme (e.g., branched peptide substrate (e.g., Z-aminobenzoyl (Abz)-Gly-Pro-Ala-Leu-Ala-4-nitrobenzyl amide (NBA), etc.), a suicide substrate, a receptor, one or more nucleotides (e.g., ATP, ADP, AMP, GTP or GDP) including analogs thereof, an oligonucleotide (e.g., a double stranded or single stranded DNA), a glycoprotein, a polysaccharide, a peptide-nucleic acid (PNA), a solid support (e.g., a sedimental particle such as a magnetic particle, a sepharose or cellulose bead, a membrane, glass (e.g., glass slides), cellulose, alginate, plastic or other synthetically prepared polymer (e.g., an eppendorf tube or a well of a multi-well plate), self-assembled monolayers, a surface plasmon resonance chip, or a solid support with an electron conducting surface), a drug (e.g., a chemotherapeutic such as doxorubicin, 5-fluorouracil, or camptosar (CPT-11; Irinotecan), etc.), a pH sensitive agent, a radionuclide, a molecule which is electron opaque, a contrast agent (e.g., barium, iodine or other MRI or X-ray contrast agent), a molecule which is sensitive to a reactive oxygen, a nanoparticle (e.g., an immunogold particle, paramagnetic nanoparticle, upconverting nanoparticle, or a quantum dot), a nonprotein substrate for an enzyme, an inhibitor of an enzyme, a chelating agent, a cross-linking group (e.g., a succinimidyl ester or aldehyde, glutathione, etc.), biotin or other avidin binding molecule, avidin, streptavidin, cAMP, phosphatidylinositol, heme, a ligand for cAMP, a metal, one or more dyes (e.g., a xanthene dye, a calcium sensitive dye, a sodium sensitive dye, a NO sensitive dye, or other fluorophore), a hapten or an immunogenic molecule (e.g., one which is bound by antibodies specific for that molecule), a radionuclide (e.g., ³H,¹⁴C,³⁵S, ¹²⁵I, ¹³¹I, etc).

In some embodiments, the functional portion of the tagging agent imparts a functionality that is useful for quantifying or detecting the presence or location of (e.g., within a cell, tissue, organism, or other system) of the lipid probe (e.g., a fluorophore, contract agent, or other detectable group). In some embodiments, the functional portion is useful for purifying or isolating the lipid probe and any other entities (e.g., proteins, complexes, etc.) stably associated therewith (e.g., bound to the activatable moiety); such functional portions include biotin, a hapten or an immunogenic molecule (e.g., one which is bound by antibodies specific for that molecule), etc. In some embodiments, the functional portion is useful in immobilization of the lipid probe and any other entities (e.g., proteins, complexes, etc.) stably associated therewith (e.g., bound to the activatable moiety); such functional portions include a solid surface (e.g., bead) or other physical handle. In some embodiments, functional portions provide a diagnostic or therapeutic functionality.

In some embodiments, activatable moieties (e.g., photoactivatable moieties) and/or taggable moieties (e.g., clickable moieties) are attached to lipid moieties via a suitable linker. In some embodiments, the functional portion and reactive portion of a tagging agent are attached via a suitable linker. In some embodiments, a linker facilitates attachment, orientation, positioning, of the various moieties. In some embodiments, a linker imparts a desirable characteristic such as solubility, insolubiliy, stability, etc. to the probe. In some embodiments, a linker is an organic group between 1.5 Å and 50 Å in length (e.g., 2 Å, 4 Å, 6 Å, 8 Å, 10 Å, 15 Å, 20 Å, 25 Å, 30 Å, 35 Å, 40 Å, 45 Å, 50 Å, or suitable ranges there between). In some embodiments, a linker is a straight or branched carbon chain of between 1 and 30 carbons (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30). In some embodiments, a linker comprises one or more heteroatom substituents (e.g., O, N, S, P, Cl, Br, F, etc.) within or appended to the carbon chain. Linkers are not limited by the type of substituents. Other suitable linkers (e.g., peptide linker, PEG linker, etc.) may be used.

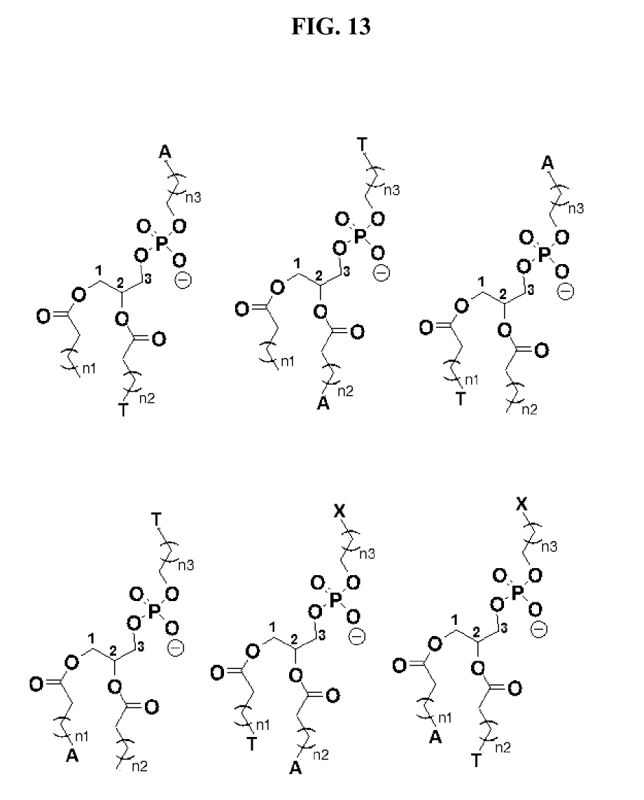

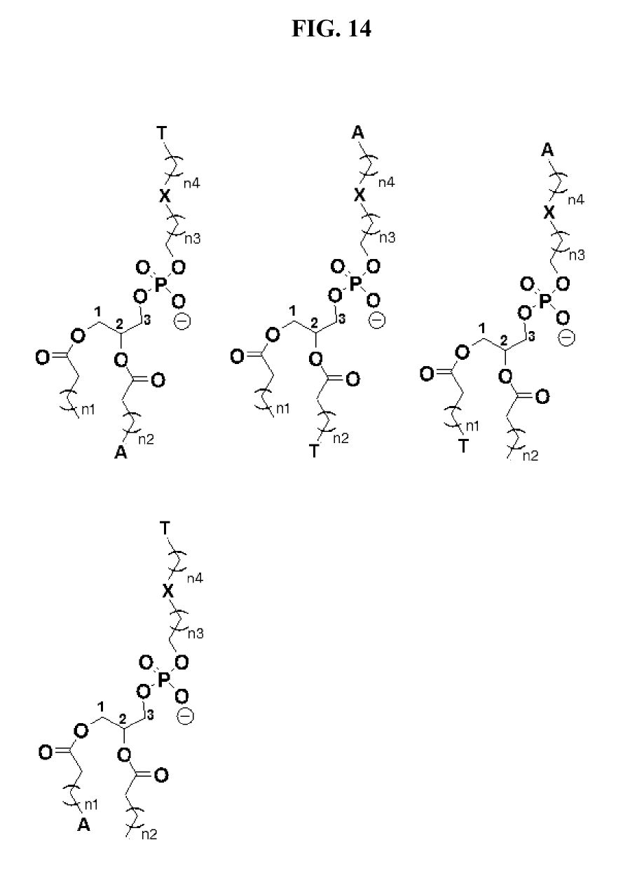

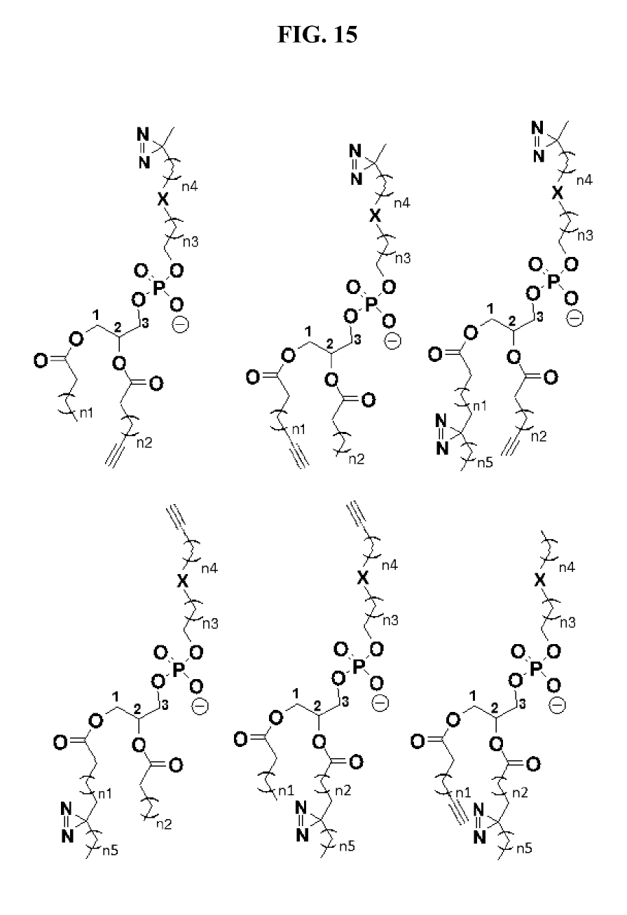

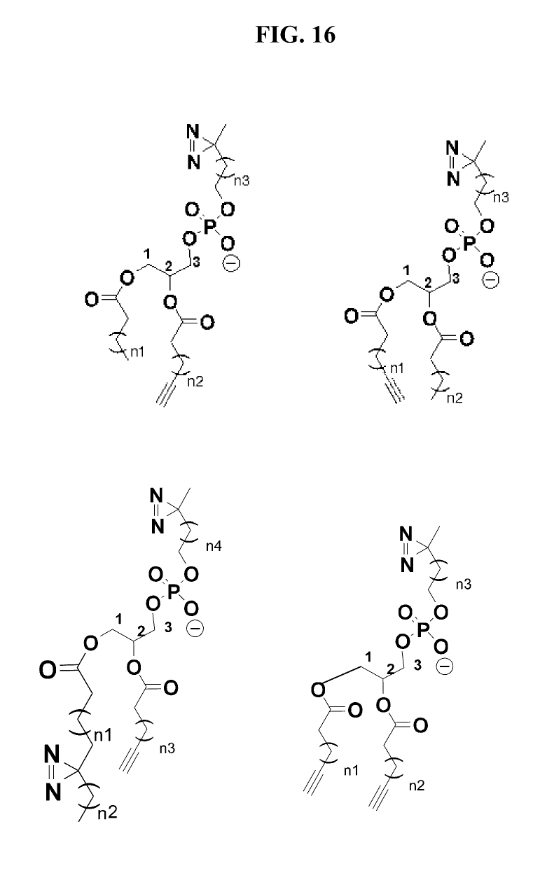

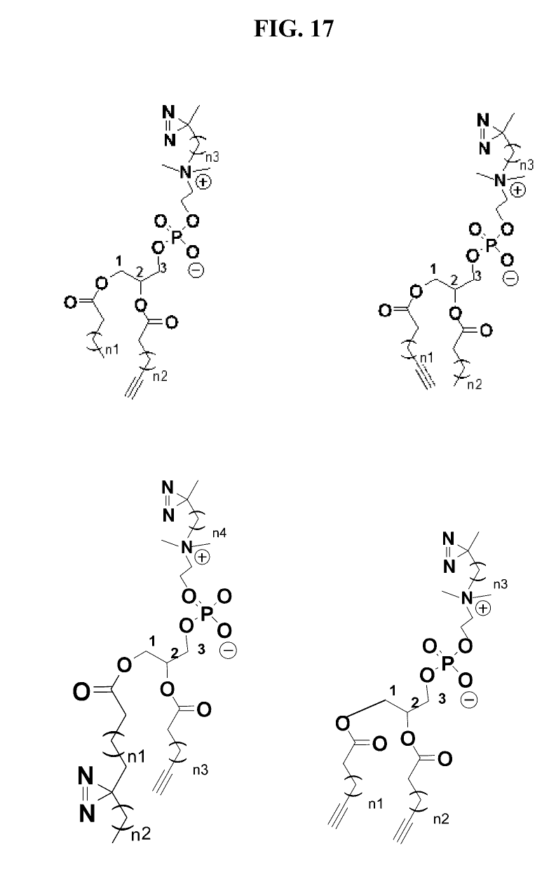

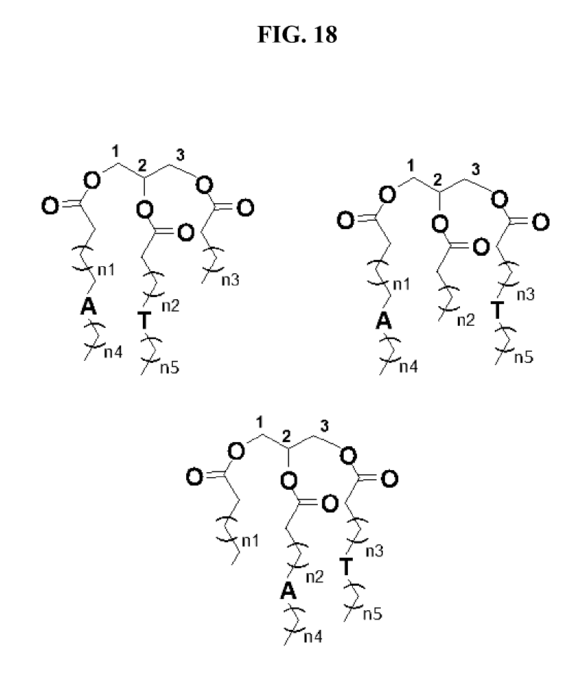

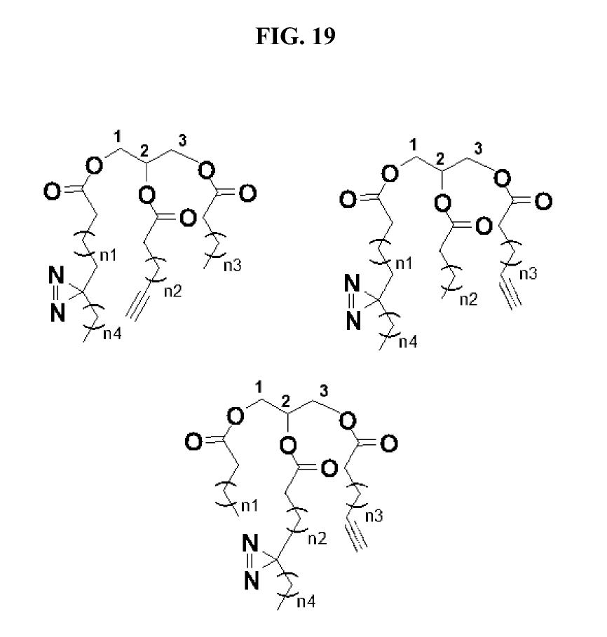

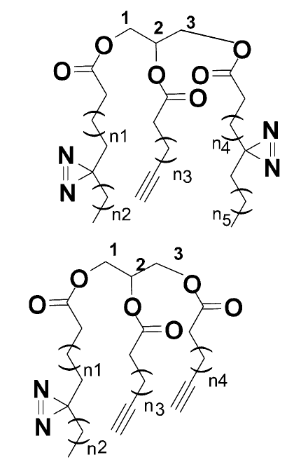

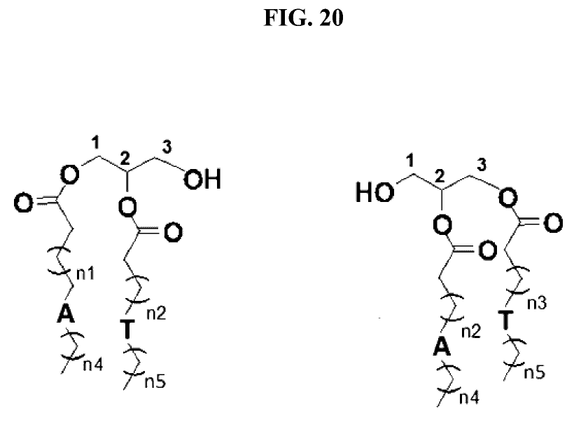

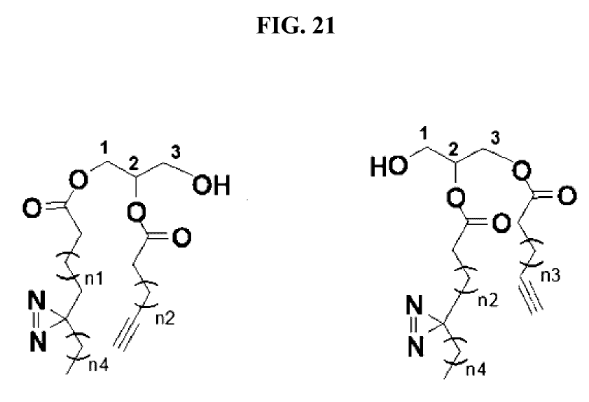

The probes described herein comprise a lipid moiety, an activatable moiety, and a taggable moiety. As addressed above, any suitable combination of lipid, activatable functional group, and taggable functional group may be assembled into a probe, and may find use in embodiments herein. FIGS. 13-21 depict particular structures and general structures highlighting embodiments of the present invention. In FIGS. 13-21, A (when present) is an activatable group; T (when present) is a taggable moiety; X (when present) is a head group (e.g., choline, serine, inositol, ethanolamine, a non-standard phospholipid head group); and n1 (when present), n2, n3 (when present), n4 (when present), and n5 (when present) are independently 0-28. Embodiments herein are not limited to the structures depicted in these figures and any other suitable assemblages of lipid, activatable functional group, and taggable functional group may be used in certain embodiments.

Beyond, or in addition to the structures in FIGS. 13-21, probe analogues are contemplated in which alternative phospholipids, lipids, sterols, etc. are used. Examples include alterations in the chain length of fatty acids (e.g., at sn-1 or sn-2 positions), switching the positions of fatty acid groups, activatable groups, and taggable groups. For example, in some embodiments, the phosphate head groups in FIG. 13 are replaced by a ceremide or sphingolipid analogue (e.g., with the taggable or activatable moiety attached thereto).

Alternative positions for the activatable moiety permit probing of the surface or interior of a membrane or other lipid-contain structure interior. In this manner, contact residues that interact with lipid surfaces, the interior or lipid membranes, or encapsulated within lipids are amenable to characterization. In some embodiments, activatable groups (e.g. diazerine ring) are placed within or along a fatty acid chain, rather than at the terminus. Such orientation of the activatable groups allows probing for contacts at varying depths within a lipid or membrane structure. This structural configuration (e.g., placing the activatable (e.g., photoactivatable) moiety on a fatty acid side chain) provides for generation of alternative classes of head groups, including a phosphatidic acid group of compounds where no head group is present.

In some embodiments, the activatable moiety (e.g., diazirine group) is placed at any suitable position along one of the fatty acyl side chains to probe residues on docking proteins that penetrate to greater depths of a hydrophobic surface. Base hydrolysis of these generates peptides adducted to the fatty acyl moiety harboring the activatable moiety (e.g., diazirine group). Incorporation onto different classes of lysophospholipid permit incorporation of the probe into phospholipids of alternative classes (head groups).

In some embodiments, probes comprise oxyester linkages between fatty acid and glycerol backbone. Yet, in alternative embodiments, the linkages between fatty acyl chains and the glycerol backbone may comprise, for example, a plasmlaogen (alkenyl linkage) linkage with a vinyl ether linkage (e.g., at the sn-1 position). Plasmalogens are abundant in the plasmolemma of cells. In some embodiments, generation of a plasmalogen analogue allows for identification and characterization of specific docking proteins (e.g., some proteins show specific binding and activity against plasmalogens vs diacylglycerophospholipids). The plasmalogen (vinylether) linkage is acid labile.

Another subclass of phsopholipids are the alkyl linkage (ether linkage) forms (e.g., at the sn-1 position of the glycerol backbone). This linkage is stable, and the mass of the base hydrolysis product formed using this form of analogue increases corresponding to the length of the fatty acid side chain at the sn-1 alkyl linkage (typically 16 or 18 carbon).

Lysophosphospholipid analogues (e.g., the sn-1 fatty acid chain is absent) are also structural analogues useful in creation of probes within the scope herein. In some embodiments, utilizing the lysophospholipid form, only the fatty acyl chain harboring the taggable moiety (e.g., alkyne) is connected to the glycerol (e.g., at sn-2 or sn-1 position).

Methods are provided comprising the use of the probes herein (e.g., along with appropriate tagging agents) for research, diagnostic, therapeutic, or other applications.

The probes herein are particularly useful as research tools for identifying and characterizing contact sites supporting protein interactions with lipid interfaces such as found on cell membranes or lipoproteins. Depending on the probes used, surface contact sites, membrane-interior contact sites, or membrane-encapsulated contact sites are probed. Further, depending on the time-scale and reactivity of the activatable group, probes may identify only close/stable contacts or may be tailored to identify looser, more transient interactions.

In some embodiments, a probe comprises multiple activatable groups, in order to allow for identification of multiple contact sites on the same lipid. Such probes also find use in identification of interactions or close contacts between proteins or other entities within lipid-containing structures.

Because the probes herein interact with (e.g., become incorporated into) various lipid- containing structures (e.g., within cells), and because the identity of the type structures with which a particular probe interacts can be engineered based on the type of lipid from which the probe is derived, the probes herein are useful in studying many (e.g., all) classes and types of lipid-containing structures. Exemplary lipid-containing structures include cell membranes, organellar membranes (e.g., nuclear membranes, mitochondrial membranes, chlorplastic membranes, lysosomes, etc.), liposomes, exosomes, lipoproteins (e.g., high-density (HDL) and low-density (LDL) lipoproteins), etc.).

Further, the probes and tagging agents herein are not limited to uses related to identifying contacts within lipid-containing structures. Because of the diversity of lipid- containing structures within which probes herein insert (e.g., a characteristic which is engineerable based on the identity of the lipid moiety of the probe), compositions herein find use in localizing diagnostic or therapeutic agents to locations on or within cells, organelles, tissues, organisms, etc., and provide useful drug/diagnostic delivery vehicles. For example, in some embodiments, the lipid moiety of a probe delivers the probe to a particular lipid-containing structure within a system, the activatable moiety allows a user to covalently link the probe to a protein of interest. The addition of the tagging moiety comprising a diagnostic or therapeutic functional portion then associates that functional portion with the protein of interest. In some embodiments, even if the protein of interest become unassociated with the original lipid-containing structure, the activatable attachment of the probe to the contacting protein (or complex) keeps the tagged probe associated.

Probe bound (e.g., and tagged) proteins, complexes, and/or other entities are immobilized, purified, identified, characterized etc. by any suitable techniques (e.g., protein analysis techniques) understood in the field. In some embodiments, the presence of the tag on the probe provides for purification or isolation of the bound proteins, complexes, and/or other entities. In some embodiments, the present of the tag on the probe provides for detection and/or characterization of the bound proteins, complexes, and/or other entities. In some embodiments, the location (e.g., amino acid residue) of the bound probe on the proteins, complexes, and/or other entities reveals the location of contact/interaction between the lipid and protein, complex, and/or other entity. In some embodiments, probing a lipid-containing structure with difference probes described herein (e.g., different fatty acids, different location of activatable moieties) enables elucidation of the nature of the interaction between the lipid and specific locations on the bound proteins, complexes, and/or other entities.

Suitable techniques for the analysis of bound proteins, complexes, and/or other entities include, but are not limited to mass spectrometry (e.g., ESI, MALDI, TOF, etc.), circular dichroism (CD) (e.g., fav-UV CD), chromatographic techniques (e.g., liquid chromatography, affinity chromatography, LC/MS, HPLC, etc.), biomolecular interaction (e.g., surface plasmon resonance (SPR)), UV-VIS absorption, fluorescence after excitation, electrochemical response, by one- or two-dimensional gel electrophoresis (e.g., SDS-PAGE), protein digestion (e.g., trypsin digestion), immunochemical analysis (e.g., immunoassay, western blotting, etc.), x-ray crystallography, nuclear magnetic resonance (NMR), etc. In some embodiments, systems and kits herein comprise reagents or components for analysis by the aforementioned techniques or other suitable methods.

In some embodiments, provided herein are systems or kits comprising the probes described herein. For example, kits/systems may be directed to identification of a peptide and/or protein interacting with a particular lipid or present in a particular lipid-containing structure, labeling of a protein within the context of a lipid-containing structure, attaching a diagnostic/therapeutic reagent to a protein within the context of a lipid-containing structure. Kits comprising the probes herein and/or that are useful in carrying out the methods herein are contemplated. Kits are an assemblage of materials or components, including at least one probe or tagging agent described herein. In some embodiments, kits comprise components or reagents for analysis of proteins following associating with a probe and tagging with a tagging agent.

The kits may include instructions for use. “Instructions for use” typically include a tangible expression describing the technique to be employed in using the components of the kit to effect a desired outcome, such as to conjugate a protein to the probes/tagging agents herein.

The materials or components assembled in the kit are provided to the practitioner stored in any convenient and suitable ways that preserve their operability, sterility and/or utility. The components are typically contained in suitable packaging material(s). The kits may maintain the operability of the reagents (e.g., probes) therein for use in, for example, proteomic analysis and identification of proteins within lipid-containing structures, or diagnostic/therapeutic uses. In some embodiments, kits comprise solid supports, antibodies, streptavidin, or any other reagents or components useful with the probes and tagging reagents herein.

In some embodiments, computer software and/or hardware is provided (e.g., as a part of a system or kit herein) for the identification (e.g., automated) of proteins, protein complexes, protein fragments, contact residues, etc.

### Experimental

Experiments were conducted to test the utility of a photoactivatable phospholipid developed as an embodiment of the present to identify, for example, protein-lipoprotein interactions. In particular, the exemplary photoactivatable phospholipid was used to identify residues in paroxonase 1 (PON1) that are involved in interactions with high density lipoprotein (HDL). The following experiments revealed that PON1 residues Leu9, Tyr185 and Tyr293 participate in HDL docking and PON1 activity, and importantly, demonstrate the power and utility of the class of photoactivatable phospholipid probes for investigating protein-lipid interactions.

### EXAMPLE 1

### Materials and Methods

### General Methods:

Solvents used in syntheses were distilled under a nitrogen atmosphere prior to use, and all materials were obtained from Sigma-Aldrich unless specified. Chromatography was performed with ACS grade solvent. Progress of synthetic reactions was monitored by thin layer chromatography, and R_{f} values are quoted for plates of thickness 0.25 mm. The plates were visualized with iodine. Flash column chromatograph was performed on 230-400 mesh silica gel supplied by E. Merck. Protein content was measured using the Markwell modified

Lowry method (ref 48; herein incorporated by reference in its entirety) with bovine serum albumin as standard. PON1 activity measurements (paraoxonase and arylesterase activities) were performed as previously described (ref 49; herein incorporated by reference in its entirety). Proton magnetic resonance (¹H NMR) spectra were recorded on a Varian Inova AS400 spectrometer operating at 600 MHz. Proton chemical shifts are reported as parts per million (ppm) on the δ scale relative to CDCl₃ (δ 7.24). ¹NMR spectral data are tabulated in terms of multiplicity of proton absorption (s, singlet; d, doublet; t, triplet; m, multiplet; br, broad), coupling constants (Hz), number of protons.

### Chemical Syntheses of 1,2-dimyristoyl-sn-glycero-3-phospho-2-(3-methyl-3H-diazirin-3-yl)ethanol

1,2-dimyristoyl-sn-glycero-3-phosphate (DMPA, 200 mg, 0.33 mmol), 2-(3-methyl-3H-diazirin-3-yl)ethanol (70 μl, 0.7 mmol) and 2,4,6-triisopropylbenzenesulfonyl chloride (TPSC1, 300 mg, 0.96 mmol) were dissolved in 10 ml anhydrous pyridine and the reaction mixture stirred overnight at room temperature. The reaction was quenched by adding 0.5 ml water. Pyridine was removed through rotary evaporation. The residue was further suspended into 70 ml cold ether (4° C.) overnight. After filtration, ether was removed by rotary evaporation. The crude product was further purified by silica gel chromatography eluted by 10% methanol in chloroform to obtain the pure product (139.4 mg, yield 63%, R_{f}=0.37 [CHCl₃/CH₃OH/H₂O=15/5/0.5)]. ¹H NMR (600 MHz, CDCl₃) δ 5.28 (br, 1H), 4.42 (br, 1H), 4.18 (br, 1H), 3.98 (br, 2H), 3.77 (br, 2H), 2.40-2.20 (4H), 1.70-1.50 (6H), 1.30-1.10 (40H), 1.03 (s, 3H), 0.85 (t, 6H). Negative ESI-MS/MS analysis yielded the following diagnostic parent and fragment ions: m/z 673.8 [M-H]⁻; m/z 645.6, 435.5, 227.6.

### Chemical Syntheses of 1-myristoyl-sn-glycero-3-phospho-2-(3-methyl-3H-diazirin-3-yl)ethanol

1,2-dimyristoyl-sn-glycero-3-phosphotidyl-2-(3-methyl-3H-diazirin-3-yl)ethanol (100 mg, 0.15 mmol) in a 50 ml centrifuge tube as dry film was suspended in 20 ml PBS buffer (10 mM, pH 8.0 and containing CaCl₂ 5 mM) and was further vortexed for 5 min. 500 μl phospholipase A₂ (PLA₂, 100 units, Sigma P6534-10 MG) was added. The resulting mixture was gently vortexed for another 2 min and incubated at 37° C. in a shaker overnight. Lipids were extracted by Bligh & Dyer method and the aqueous phase was further extracted with chloroform (10 ml×2). Organic solvents were combined and evaporated. Crude product was further purified by silica gel column eluted with 15% methanol in chloroform to obtain the pure product (45 mg, yield 65%, R_{f}=0.2, 25% methanol in chloroform). Negative ESI-MS/MS analysis yielded the following parent and diagnostic fragment ions: m/z 463.7 [M-H]⁻; m/z 435.6, 365.5, 227.7.

### Chemical syntheses of 1-myristoyl-2-(10-undecynoyl)-sn-glycero-3-phospho-2-(3-methyl-3H-diazirin-3-yl)ethanol (pac-PC)

1-myristoyl-sn-glycero-3-phosphotidyl-2-(3-methyl-3H-diazirin-3-yl)ethanol (20 mg, 0.043 mmol), 10-undecynoic acid (40 mg, 0.22 mmol), N,N′-dicyclohexylcarbodiimide (DCC, 26 mg, 0.126 mmol) and 4-dimethylaminopyridine (DMAP, 5 mg, 0.043 mmol) were dissolved in 2 ml anhydrous chloroform and stirred at room temperature for 48 h. Solvents were removed by rotary evaporation. Crude product was further purified by silica gel column eluted with 10% methanol in chloroform to deliver the final product (20.4 mg, yield 75%, R_{f}=0.36 with 20% methanol in chloroform). ¹H NMR (600 MHz, CDCl₃) δ 5.28 (br, 1H), 4.35 (br, 2H), 4.14(br, 2H), 4.02 (br, 1H), 3.82 (br, 1H), 2.40-2.20 (4H), 2.20-2.10 (2H), 1.92 (m, 1H), 1.70-1.55 (6H), 1.50 (m, 2H), 1.30-1.10 (28H), 1.04 (s, 3H), 0.86 (t, 3H). Negative ESI-MS/MS analysis yielded the following diagnostic parent and fragment ions: m/z 627.7 [M-H]⁻; m/z 599.5, 435.5, 228.3, 182.1.

Preparation of Reconstituted Nascent HDL Containing pac-PC:

All human biological materials were obtained from participants that gave written informed consent for protocols approved by the Cleveland Clinic Institutional Review Board. Human HDL was isolated from plasma of healthy volunteers by sequential buoyant density ultracentrifugation using KBr to adjust density to the range of 1.07 g/ml-1.21 g/ml, as previously described (refs. 50-52; herein incorporated by reference in their entireties). Lipid free human apoA-I was prepared by delipidation of isolated human HDL using methanol/ether/chloroform followed by ion exchange chromatography (ref 53; herein incorporated by reference in its entirety). The purity of isolated human apoA-I was verified by SDS-PAGE. Reconstituted nascent HDL (rHDL) was prepared using modified sodium cholate dialysis method (refs. 53-54; herein incorporated by reference in their entireties). An initial molar ratio of 98/2/10/1 of 1-palmitoyl-2-oleoyl sn-glycero-3-phosphocholine (POPC): pac-PC: cholesterol: isolated human apoA-I was used for reconstituted HDL preparation.

Photoactivatable Cross-Linking between pac-PC and Human apoA-I or PON1

(i) the cross-linking between pac-PC and human apoA-I: rHDL with pac-PC (human apoA-I conc. 70 μM) in 0.5 ml of 50 mM PBS buffer at pH 7.0 was transferred into small glass vials on ice and then directly exposed to 365 nm UV light for 1 min under UV lamp. Proteins were then precipitated by adding 9 ml methanol/chloroform/ether, 1/1/2.5, (v/v/v).

(ii) the cross-linking between pac-PC and PON1: rHDL with pac-PC (human apoA-I conc. 70 μM) in 0.5 ml of 50 mM PBS buffer at pH 7.0 was incubated with recombinant His-tagged PON1 (rPON1, 30 μM, in 0.5 ml 50 mM Tris buffer with 1 mM CaCl₂, at pH 7.0) that was expressed and purified from E. Coli (ref 55; herein incorporated by reference in its entirety). The mixture was incubated at 37° C. for 1 hour. The mixture was then transferred into small glass vials on ice and then directly exposed to 365 nm UV light for 1 min under UV lamp. Proteins were then precipitated by adding 18 ml methanol/chloroform/ether, 1/1/2.5, (v/v/v). Precipitated proteins (mixture of apoA-I and PON1) were further dissolved into 1 ml PBS buffer (50 mM, pH 7.0) with 1% SDS, and further diluted by addition of 4 ml PBS buffer (to reduce SDS concentration to 0.2%). Nickel nitrilotriacetic acid resin (Ni-NTA, 500 ul, pre-washed and equilibrated in PBS buffer, 50 mM, pH 7.0), was added to enrich His-tagged PON1 and to remove human apoA-I. rPON1 was further eluted with PBS buffer (50 mM, pH 7.0) containing 0.2% SDS and 200 mM imidazole. rPON1 was further precipitated by using methanol/chloroform/ether, 1/1/2.5, (v/v/v).

Adding Biotin Tag to pac-PC Cross-Linked Proteins through Click Chemistry:

Precipitated protein (apoA-I or rPON1) that had been cross-linked with the pac-PC was further dissolved in 1 ml PBS buffer (50 mM, pH 7.0 and with 1% SDS). Biotin-azide (ref. 56; herein incorporated by reference in its entirety) (4 μl, 30 mM in dimethylformamide) was added and the mixture was vortexed for 1 min. Then, fresh made tris(2-carboxyethyl)phosphine (TCEP, 20 μl, 50 mM in water) was added, followed by the addition of tris[(1-benzyl-1H-1,2,3-triazol-4-yl)methyl]amine (TBTA, 20 μl, 5 mM in DMSO: t-butanol=1 :4) and the mixture was vortexed for another 1 min. The reaction was initiated with CuSO₄ (50 μl 20 mM stock solution in water) and the mixture was vortexed for 1 min. The reaction mixture was left at room temperature for 1 hour (vortex after 30 min). Reaction product was further transferred into slide-a-lyzer dialysis cassettes (Thermo scientific) and dialyzed in 4 liter PBS buffer at 4° C. (change buffer at least 3 times in 24 hours).

Affinity Purification of pac-PC Modified Peptides:

The affinity purification of pac-PC cross-linked proteins and subsequent on-bead trypsin/chymotrypsin digestion steps were performed using the general tandem orthogonal proteolysis strategy (ref 57; herein incorporated by reference in its entirety). Photoactivatable lipid probe (pac-PC) cross-linked, and biotin tagged proteins (apoA-I or rPON1) were incubated with Neutravidin beads (200 μl, 1/1 slurry, pre-washed and equilibrated in PBS buffer) for 3 h at room temperature. Supernatant was removed by centrifugation at 1,400 g for 3 min. Neutravidin beads were washed by 5 ml PBS (with 0.2% SDS) three times and followed by 5 ml water. Beads were transferred into a low-retention Eppendorf tube and incubated with a premixed solution (200 μl of 1 M urea/PBS, 2 μl of 1 M calcium chloride in water and 4 μl of 0.5 μg/μl trypsin or chymotrypsin) at 37° C. overnight with rotations. Neutravidin beads were further washed with PBS buffer (3×500 μl) and water (3×500 μl). Beads were then treated with 50/50 (v/v) water/acetonitrile containing 0.1% trifluoroacetic acid (200 μL, 65° C., 5 min) (ref 58; herein incorporated by reference in its entirety). Beads were removed via filtration through a spin column and washed with 50/50 (v/v) water/acetonitrile (2×100 μl) for a total elution volume of 400 μl. The solvent was evaporated under N₂ flow to a final volume ˜25 μl and was further diluted by another 25 μL 0.1% formic acid/water. For the base treatment, sodium hydroxide (1 M in water, 5 μl) was added and the resulting mixture was incubated at room temperature for 3 h. Then acetic acid (2 M in water, 3 μl) was added to neutralize the sample. The samples were further analyzed by liquid chromatography with on-line electrospray ionization tandem mass spectrometry (LC/MS/MS).

### Mass Spectrometry Analysis:

All mass spectrometry analyses were performed under Xcalibur data system using Proxeon Easy-nLC coupled to a LTQ Orbitrap Velos mass spectrometer (Thermo Scientific) with a nano-LC electrospray ionization source. Samples were first pressure loaded onto an IntegraFrit sample trap (ID 100 um, length 2.5 cm, New Objective) and further separated by a PicoFrit column (stock# PF360-75-15-N-5, New Objective) packed in our laboratory (pacing material: C18, 300 Å, 5 μm from Cobert Associates). Mobile phase: Buffer A: water with 0.1% formic acid; Buffer B: acetonitrile with 0.1% formic acid). The HPLC gradient started with 5% B and increased to 40% B over 60 min, then increased to 80% B over 5 min, held at 80% B for another 10 min. For repeat injections on autosampler, the column was reequilibrated for next sample by decreasing to 5% B over 2 min and held at this solvent composition for 20 min. Flow rate was 0.2 μl/min. The applied spray voltage of the nano-LC electrospray ionization source is 2.0 kV. For MS₂ data collection, one full scan by Orbitrap was followed by 10 data dependent scans of the most intense ions by ion trap. Dynamic exclusion was also applied so that any ions that had been repeatedly scanned three times in 60 seconds would be excluded for further scan for 3 minutes.

Mass spectra were subject to SEQUEST (ref 59; herein incorporated by reference in its entirety) (Thermo Scientific) and searched against a PON1.fasta database. A static modification +926.532, corresponding to the lipid probe, was applied to each amino acid residue. SEQUEST output files were filtered by XCorr (cross-correlation value, min XCorr=1.8(+1), 2.5(+2), 3.5(+3) as described in the DTASelect (ref 60; herein incorporated by reference in its entirety)). For base treated samples, a static modification of +226.061 corresponding to a glycerol phosphate group was used. Other possible modifications were also applied during the SEQUEST search, including oxidation on methionine (+16), deamination of glutamine and asparagine (+1) and dehydration of serine and threonine (−18). All targeted peptides spectra were examined by manual inspection.

Site-Specific Mutations of Recombinant Paraoxonase 1 (rPON1):

Site specific mutants of rPON1 used included the G3C9 PON1 clone (ref 55; herein incorporated by reference in its entirety) on the wild type (WT) backbone sequence. Pan E PON1 mutant (Leu9→Glu9, Tyr185→Glu185 and Tyr293→Glu293) and pan K PON1 mutant (Leu9→Lys9, Tyr185→Lys185 and Tyr293→Lys293), with a His₈ tag directly at their carboxyl terminus, were prepared from codon optimized WT rPON1 directly synthesized by GenScript (Piscataway, N.J.) and cloned at NcoI-HindIII restriction sites into the pBAD vector (Invitrogen). Competent TOP10 (Invitrogen) E. coli were transfected with the plasmids for the expression of the recombinant proteins. Wild type and mutant rPON1s were purified by Ni-NTA affinity chromatography to homogeneity, with purity of proteins confirmed by SDS-PAGE.

Spectral Studies of rPON1 Mutants:

Far-UV circular dichroism spectra were recorded on a Pistar180 spectropolarimeter (Applied Photophysics, Surrey, UK). Standardization was performed with an aqueous solution of 0.06% ammonium d-(+)-10-camphor sulfonate at a wavelengths of 260 to 200 nm and with a path length of 10 mm. rPON1 forms (WT, pan E and pan K mutants) were prepared as 0.1 mg/ml samples in 10 mM phosphate buffer, pH 7.0 supplemented with 0.2 mM CaCl₂, analyzed at ambient temperature in continuous scan mode with a 1-nm bandwidth (100000 counts/step).

### Surface Plasmon Resonance (SPR):

Full time course kinetic determination of kₒₙ, k_{off} and apparent equilibrium dissociation constants K_{d} for rHDL interaction with rPON1 forms were determined using plasmon resonance spectroscopy (SPR) using a BIAcore 3000 SPR biosensor (BIAcore, AB). rHDL was directly immobilized on a CM5 sensor chip. To determine the K_{d} between distinct rPON1 and rHDL, rPON1 ranging from 500 nM to 2000 nM were prepared in PON1 activity buffer (50 mM Tris, 50 mM NaCl, 1 mM CaCl₂, pH 8.0) and flowed over the surface of the sensor chip at a rate of 20 μl/min. At the end of each cycle, surfaces of the sensor chips were regenerated by injection of 15 mM HCl at the same flow rate. Apparent K_{d} were obtained by fitting background-subtracted SPR binding data to the 1:1 binding with drifting baseline model within the BIA evaluation software version 4.0.

Stability Studies of rPON1 Mutants:

rPON1 was pre-incubated with rHDLs (2 equivalence) or 0.1% tergitol (NP-10, Sigma-Aldrich) in PON1 activity buffer (50 mM Tris, 50 mM NaCl and 1 mM CaCl₂, pH 8.0) at 37° C. for 30 min. Inactivation was initiated by adding an equal volume of inactivation buffer (10 mM EDTA and 20 mM β-mercaptoethanol in 50 mM Tris, pH 8.0) and incubating the samples at 37° C. Aliquots were taken at various time points and the arylesterase activity was examined as described previously (ref 49; herein incorporated by reference in its entirety).

### EXAMPLE 2

### Results

### Photoactivatable Lipid Probe Design and Chemical Synthesis.

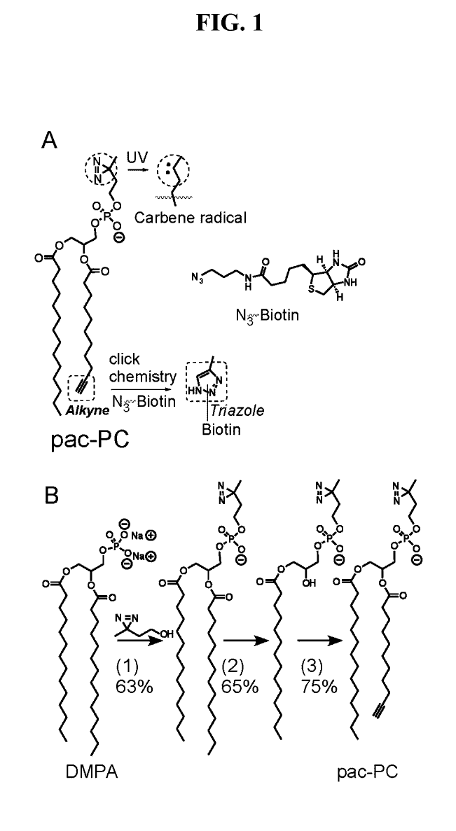

Experiments were conducted during development of embodiments herein to design and synthesize and exemplary photoactivatable and taggable probe within the scope of embodiments. To this end a photoactivatable and clickable phosphatidylcholine probe (pac-PC; FIG. 1A) was prepared. An analogue of phosphatidylcholine, the most abundant class of phospholipid in most lipoproteins and membranes, pac-PC harbors a diazirine ring that replaced the trimethylamine moiety of the choline polar head group. Upon exposure to ultraviolet (UV, 365 nm) radiation, the diazirine ring readily expels N₂ generating a reactive diradical carbene species. Carbenes rapidly insert into proximate protein back bones or side chains in nanoseconds, and if not are scavenged by water at near the diffusion-limited rate. Thus, this exemplary probe design ensures that detection of a covalent adduct between the phospholipid probe and a protein indicates existence of a tight enough binding interaction to exclude water (ref 61; herein incorporated by reference in its entirety). In addition, to facilitate subsequent affinity isolation of adducts, a terminal alkyne group was introduced on a sn-2 position aliphatic chain, providing a useful handle for the later addition of a tag (e.g., biotin tag) through highly efficient and selective click chemistry (FIG. 1A) (refs. 62,63; herein incorporated by reference in their entireties).

The chemical synthesis of pac-PC (See, FIG. 1B) began from a commercially available lipid (1,2-dimyristoyl-sn-glycero-3-phosphate, DMPA). Briefly, after being coupled with 2-(3-methyl-3H-diazirin-3-yl)ethanol (ref 64; herein incorporated by reference in its entirety), the sn-2 side chain was removed through phospholipase A₂ (PLA₂) catalyzed hydrolysis (ref. 65; herein incorporated by reference in its entirety). Further reaction of the lysolipid with 10-undecynoic acid in the presence of coupling reagents dicyclohexylcarbodiimide (DCC) and 4-dimethylaminopyridine (DMAP) delivered the final product, pac-PC (ref 66; herein incorporated by reference in its entirety). The synthesized lipid probe was further purified by HPLC and extensively characterized by nuclear magnetic resonance (NMR) and mass spectrometry to confirm its purity and validate its structure.

Photoactivatable Lipid (pac-PC) Cross-Linked with apoA-I in HDL.

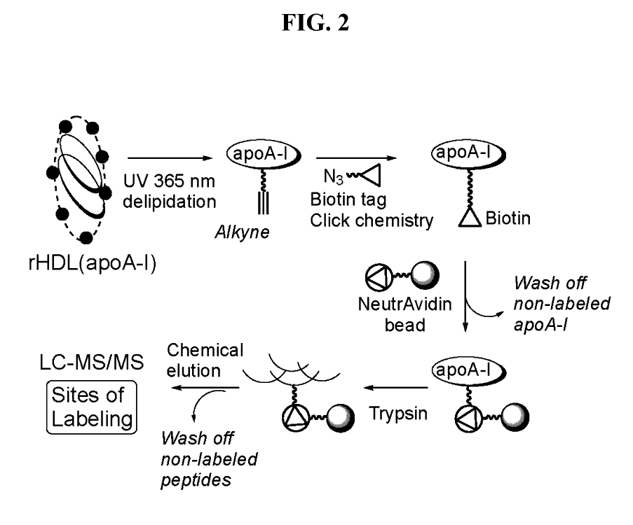

Since apoA-I interacts with phospholipids in nascent HDL particles, the interaction of pac-PC with apoA-I in HDL was initially examined to develop the analytical and mass spectrometry methods. The experimental scheme used to study lipid-protein interaction in nascent HDL is outlined in FIG. 2. Reconstituted nascent HDL (rHDL) was prepared using a modified sodium cholate dialysis method containing a low mole percent of the photoactivatable lipid probe, pac-PC. Control studies (e.g., equilibrium native PAGE and dual beam light scattering) showed that the rHDL particles thus generated had similar size compared with rHDL lacking the probe. Following UV irradiation, the pac-PC adduct was biotinylated using click chemistry. Tagged apoA-I was then subject to Neutravidin enrichment and on-bead trypsin digestion. Following a wash step, pac-PC modified apoA-I peptides were eluted from the beads, and then analyzed by LC/MS/MS.

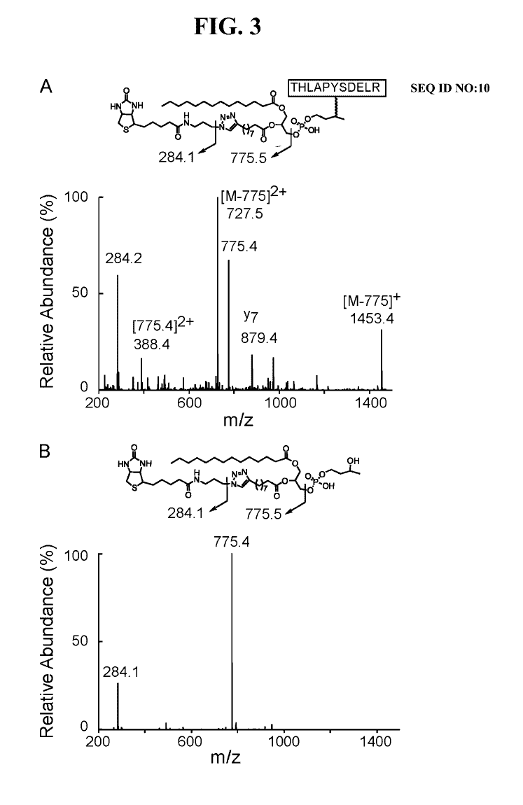

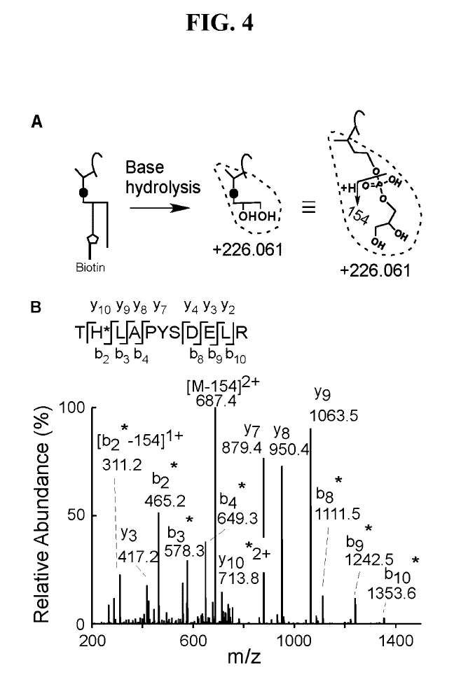

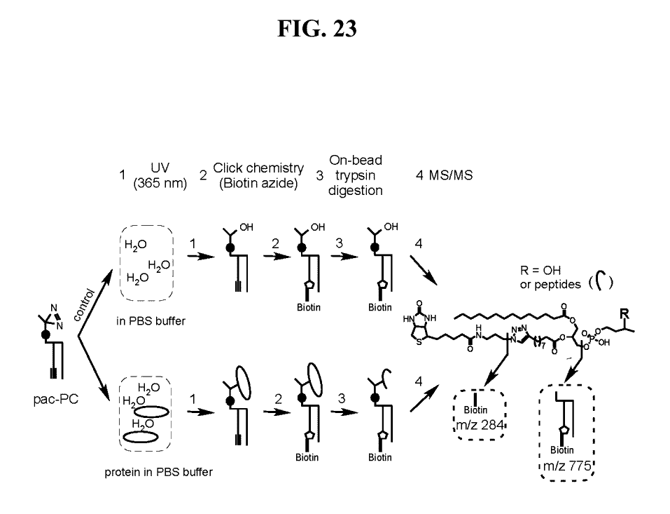

Despite initial efforts to deconvolute the raw spectra with SEQUEST searches, no modified peptides were identified. However, the manual inspection of the raw peptide spectra, identified spectra with specific daughter fragmentation, such as m/z 284 and m/z 775 (FIG. 3A). Identical daughter fragmentations (m/z 284 and m/z 775) from different peptide spectra indicated that the daughter ions at m/z 284 and m/z 775 were derived from the same source, the lipid modification. Acontrol experiment was performed as illustrated in FIG. 23. The synthetic lipid analogue pac-PC, was either incorporated into HDL (i.e. in the presence of protein) as outlined above, or incubated alone in buffer (PBS) and then exposed to UV irradiation. Samples were then incubated with biotin azide to enable click chemistry, based biotinilation, followed by on-bead trypsin digestion and LC/MS/MS analysis (FIG. 4). The pac-PC sample exposed to UV irradiation alone in buffer (without the presence of apoAl/HDL) gave m/z 284 and m/z 775 as major fragments, confirming that these daughter ions arise from the pac-PC moiety itself. Thus, the reasons for difficulty using the automated peptide sequencing was because the lipid modified peptides gave dominant fragments from cleavage of the lipid instead of from the peptide back bone, complicating spectra interpretation and lead to the software failed searches.

To circumvent this problem, a base hydrolysis step was added to the end of the overall scheme depicted in FIG. 23, the rationale for which is illustrated in the Scheme shown in FIG. 4A. The ester bonds in lipids are more base sensitive than amide bonds in peptides; thus, base-catalyzed hydrolysis effectively removed the bulk of the lipid side chains leaving a characteristic small modification group (glycerol phosphate, +226.061 amu). For base treated samples we therefore applied a static modification of +226.061 amu to each amino acid for peptide analyses, corresponding to a glycerol phosphate group, as described under Experimental Procedures. It was determined that applying this stastic +226.061 amu modification gave structurally informative and recognizable peptide backbone fragmentations (FIG. 4). Thus, base treated peptides were easily identified, and recognized with exact modification sites, by SEQUEST (FIG. 4).

Use of rHDL Incorporating pac-PC to identify PON1 Residues Involved in Binding to the HDL Lipid Surface.

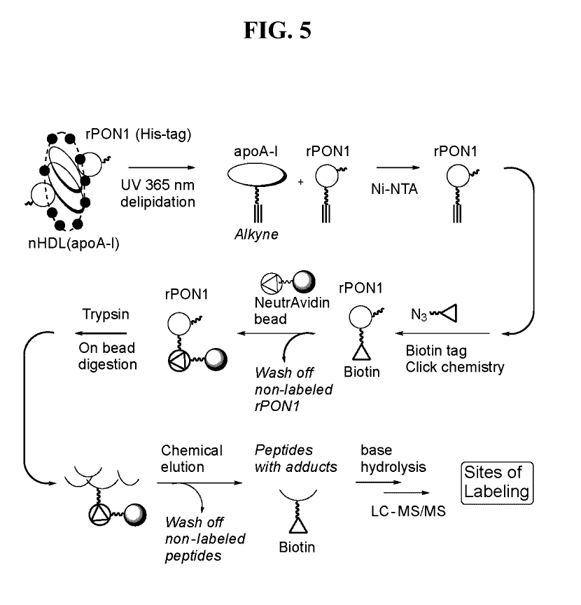

The overall experimental scheme used for the analysis of HDL-PON1 interactions is outlined in FIG. 5. Reconstituted nascent HDL (rHDL) incorporating the 2 mol % pac-PC was prepared and then incubated with His tagged rPON1 at 37° C. for 1 hour. Cross-linking was performed with brief (1 min) exposure to UV (365 nm) at 0° C. Proteins were precipitated, and then resolubilized rPON1 was enriched using Ni-NTA resin to remove human apoA-I. Immobilized PON1 containing phospholipid adducts was biotinylated via click chemistry and then lipid tagged rPON1 was subjected to Neutravidin enrichment and on-bead trypsin/chymotrypsin digestion (FIG. 5). Following a wash step to remove non-lipid adducted and biotinylated peptides, the lipid modified rPON1 peptides were eluted from the beads. After base treatment, facile identification of the modification sites on rPON1 was achieved by LC/MS/MS and searching for glycerol-phosphate modified peptides, which were detected by a static modification (+226.061) applied to each amino acid residue.

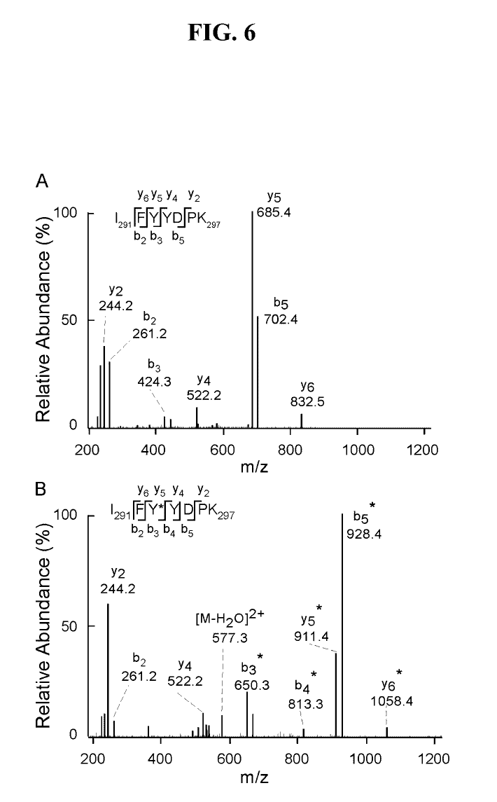

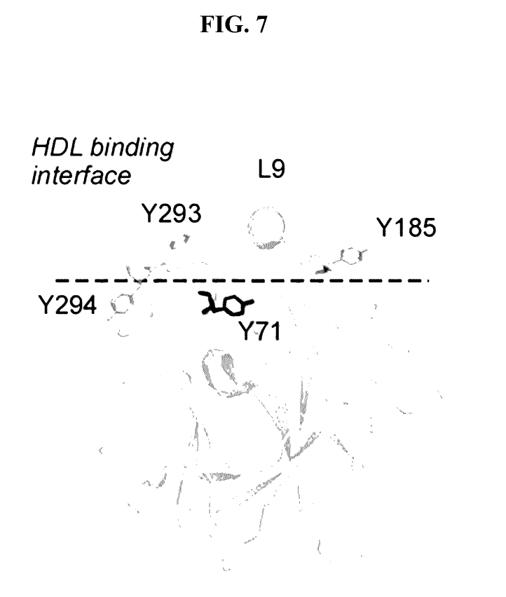

Three rPON1 peptides (Table 1) were reproducibly identified that carried the specific modification (+226.061) corresponding to the glycerol phosphate adduct (Ala₂-Lys₂₁, Ile₂₉₁-Lys₂₉₇, and Ala₁₈₀-Tyr₁₉₀). The residue within each peptide, (Leu9, Tyr185 and Tyr293) carrying the glycerol-phosphate moiety derived from pac-PC are also shown, along with the excellent (within 10 ppm) agreement between the exact mass experimentally measured for each peptide and its theoretical mass based on elemental composition (Table 1). For illustrative purposes, the mass spectra of the native peptide Ile₂₉₁-Lys₂₉₇ and the peptide Ile₂₉₁-Lys₂₉₇ carrying the modification at residue Tyr293 are presented in FIG. 6A and 6B, respectively (a static mass shift of +226 is observed on related b and y ions labeled with * in FIG. 6). Also shown is the crystal structure for PON1 (ref 68; herein incorporated by reference in its entirety) with superimposed locations of Leu9, Tyr185 and Tyr293, which collectively identify the interfacial surface that binds to the HDL particle (FIG. 7). PON1 Tyr71, a residue previously showed to be in close spatial proximity to cholesterol within PON1 bound to an HDL particle using a photoaffinity labeled cholesterol analogue (ref 29; herein incorporated by reference in its entirety), is similarly localized within the identified lipid interfacial binding surface (FIG. 7). Further, PON1 Tyr294, despite being within ˜5 Å with Tyr293 (alpha carbon to alpha carbon; FIG. 7), failed to be labeled by the pac-PC probe in PON1-bound HDL, illustrating the remarkable specificity of residue interaction and covalent modification by the phospholipid photoactivatable group in the PON1-HDL complex.

TABLE 1
Identified rPON1 residues that cross-linked with the photoactivatable lipid
probe pac-PC. Three rPON1 peptides were reproducibly identified that carried the specific
modification (+226.061) corresponding to the glycerol phosphate adduct. The residue within
each peptide, (Leu9, Tyr185 and Tyr293) carrying the glycerol-phosphate moiety derived
from pac-PC are also shown, along with the excellent (within 10 ppm) agreement between
the exact mass experimentally measured for each peptide and its theoretical mass based on
elemental composition. Mass spectra were originally filtered by XCorr (cross-correlation
value) and were further examined by manual inspection.

| Labeled peptide | Labeled residue | [M + H]⁺ | ΔM(ppm) | X-Corr |
|---|---|---|---|---|
| A₂KLTALTL*LGLGLALFDGQK₂₁ | L9 | 2269.2962 | 4.70 | 4.57 |
| (SEQ ID NO: 1) | | | | |
| I₂₉₁FY*YDPK₂₉₇ | Y293 | 1171.5417 | 4.18 | 2.29 |
| (SEQ ID NO: 2) | | | | |
| A₁₈₀TNDHY*FADPY₁₉₀ | Y185 | 1539.6117 | 5.07 | 2.51 |
| (SEQ ID NO: 3) | | | | |

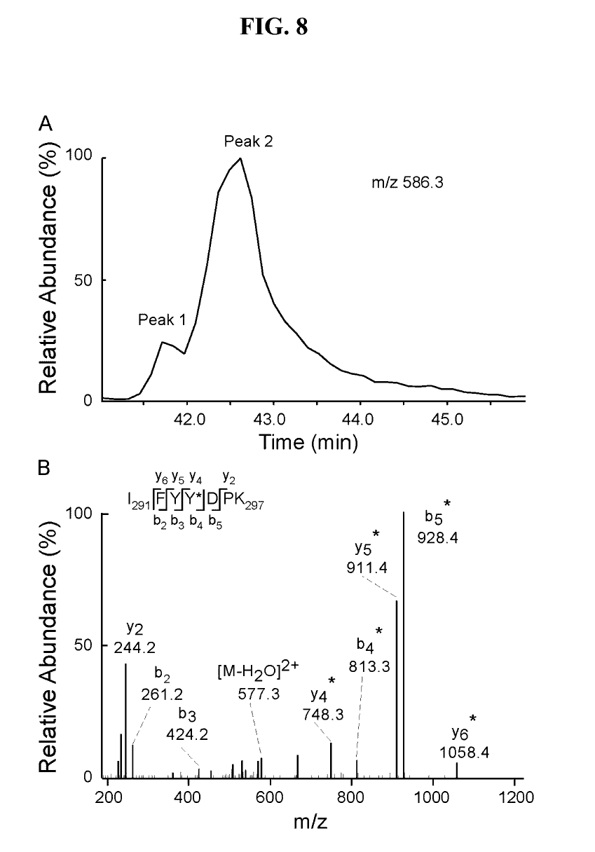

The rPON1 variant used in the experiments conducted during development of embodiments herein, G3C9, has two adjacent tyrosine residues (Tyr293 and Tyr294) in peptide Ile₂₉₁-Lys₂₉₇, but sequence analyses of affinity isolated peptides recovered from PON1-HDL complexes following photoactivation of the pac-PC probe repeatedly showed only PON1 Tyr293 was covalently adducted to the probe. To further confirm that the lipid cross-link was at Tyr293 instead of Tyr294, more sensitive analysis of selected ion monitoring (m/z 586.3, corresponding to the lipid cross-linked peptide Ile₂₉₁-Lys₂₉₇) was performed, which allowed for successful isolation of a peptide that has the same m/z value but slightly different retention time, shown as the small shoulder peak (peak 1) on the chromatogram (FIG. 8A). This less abundant peptide, Ile₂₉₁-Lys₂₉₇, was observed to carry the lipid modification at Tyr294 instead of Tyr293, based on its MS/MS fragments (FIG. 8B; adduct at Tyr 294 is estimated to occur at ˜20-fold reduced level compared to adduct at Tyr293).

PON1 residues Leu9, Tyr185 and Tyr293 are Functionally Important for HDL Binding and Preservation of PON1 Activity.

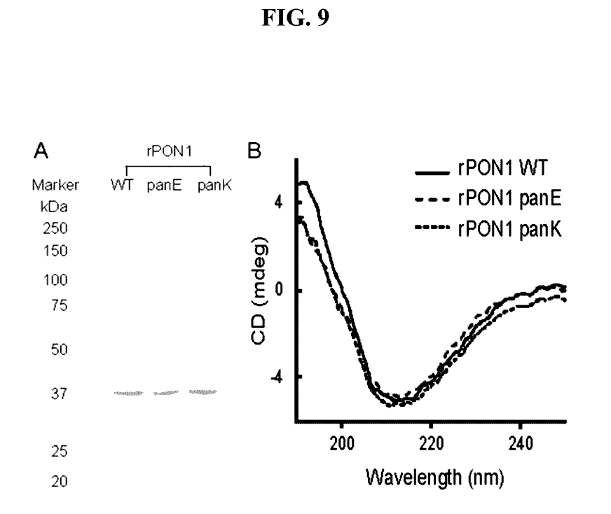

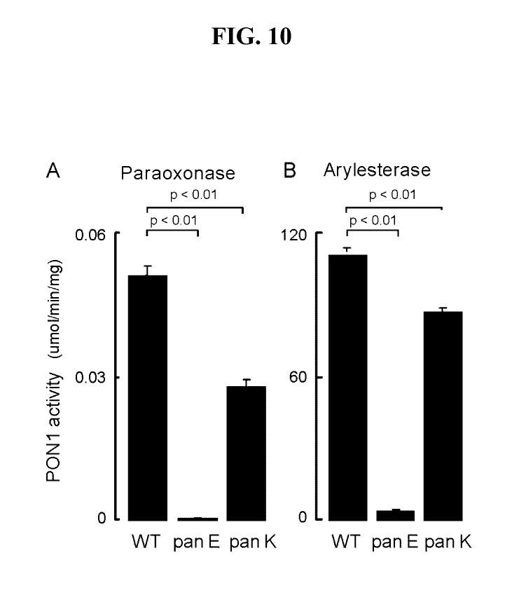

To examine the functional significance of PON1 residues discovered as HDL lipid binding sites (Leu9, Tyr185 and Tyr293), site-specific rPON1 mutants were generated. Mutants were compared with the recombinant PON1 variant G3C9 (wild type like activity, rPON1 WT). Residues (Leu9, Tyr185 and Tyr293) were mutated to either glutamic acid (rPON1 pan E) or lysine (rPON1 pan K). The purified rPON1 mutants (pan E or pan K mutants) were observed to be homogeneous and have similar molecular weight, as shown by SDS-PAGE (FIG. 9A). Moreover, secondary structure analyses, as shown by circular dichroism (CD) spectra, revealed minimal differences when compared to WT rPON1 (FIG. 9B). The catalytic activities of WT rPON1 and these rPON1 mutants were examined using paraoxon (paraoxonase activity) or phenyl acetate (arylesterase activity) as substrates. Purified rPON1 pan K mutant demonstrated a specific activity of ˜50% paraoxonase activity, and ˜80% arylesterase activity compared with rPON1 WT, whereas rPON1 pan E mutant demonstrated almost complete loss of activity with either substrate (FIG. 10A,B).

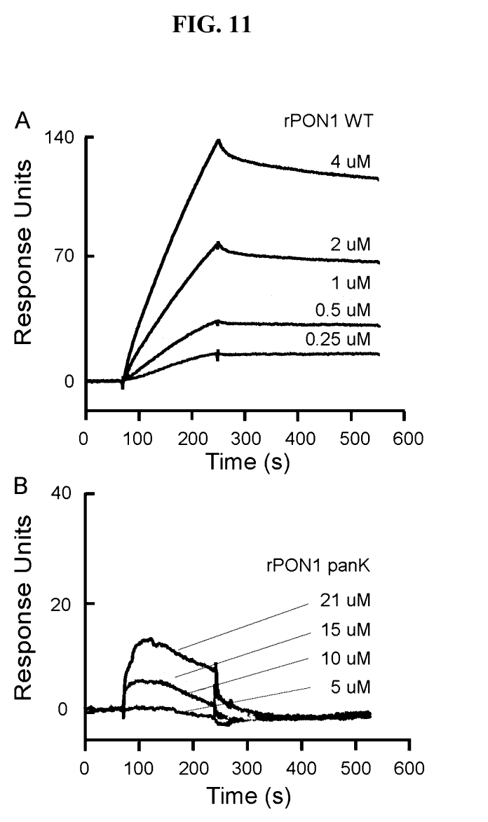

The functional significance of PON1 residues Leu9, Tyr185 and Tyr293 in HDL docking interactions were further interrogated by directly quantifying the collective role of the PON1 residues in binding to the HDL particle surface. For these studies, the binding of WT rPON1 (G3C9) and both mutant rPON1 forms (pan E and pan K) with rHDL were examined using surface plasmon resonance spectroscopy. Reconstituted HDL particles were directly coated on the sensor chips and then diluted rPON1 solutions of different concentrations were individually flowed over the immobilized rHDL. Measured Kₒₙ, K_{off} and K_{d} for each PON1 form interaction with rHDL are shown in Table 2, and for illustrative purposes, typical binding sensorgrams for binding of rPON1 WT or rPON1 pan K to rHDL are presented in FIGS. 11A and B, respectively. The rPON1 pan K mutant showed 1000-fold less binding capacity to rHDL compared with rPON1 WT (K_{d}:1.4×10⁻⁶ vs. 7.1×10⁻¹⁰ respectively, Table 2). Further, the rPON1 pan E mutant showed even lower HDL binding capacity compared to rPON1 pan K mutant (K_{d}:5.9×10⁻⁵ versus 1.4×10⁻⁶ respectively, Table 2). These results show that PON1 Leu9, Tyr185 and Tyr293 participate in PON1-HDL interactions, and the nature of the site-specific mutations impact the affinity of the PON1 form to bind to HDL.

TABLE 2
The binding affinities between rPON1 mutants (WT, pan E, pan K) and
recombinant nascent HDL particles through surface plasmon resonance. Recombinant
nascent HDL was directly immobilized on a CM5 sensor chip. Samples of the indicated
rPON1 ranging from 500 nM to 2000 nM were prepared in PON1 activity buffers (50 mM Tris,
50 mM NaCl, 1 mM CaCl₂, pH 8.0) and flowed over the surface of the sensor chip at a
flow rate of 20 μl/min. The association constant rate (Kₒₙ), dissociation constant
rate (K_{off}) and apparent dissociation constants (K_{d}) were listed in the table.

| rPON1 | Peptides with mutated amino acids | Kₒₙ (1/Ms) | K_{off} (1/s) | K_{d} (1/M) |
|---|---|---|---|---|
| WT | A₂KLTALTLLGLGLALFGGQK₂₁ _{(SEQ ID NO: 1)} | 1.2 × 10³ | 8.5 × 10⁻⁷ | 7.1 × 10⁻¹⁰ |
| | I₂₉₁FYYDPK₂₉₇ _{(SEQ ID NO: 2)} | | | |
| | A₁₈₀TNDHYFADPY₁₉₀ _{(SEQ ID NO: 3)} | | | |
| pan E | A₂KLTALTELGLGLALFDGQK₂₁ _{(SEQ ID NO: 4)} | 850 | 5.0 × 10⁻² | 5.9 × 10⁻⁵ |
| | I₂₉₁FEYDPK₂₉₇ _{(SEQ ID NO: 5)} | | | |
| | A₁₈₀TNDHEFADPY₁₉₀ _{(SEQ ID NO: 6)} | | | |
| pan K | A2KLTALTKLGLGLALFDGQK₂₁ _{(SEQ ID NO: 7)} | 42 | 6.0 × 10⁻⁵ | 1.4 × 10⁻⁶ |
| | I₂₉₁FKYDPK₂₉₇ _{(SEQ ID NO: 8)} | | | |
| | A₁₈₀TNDHKFADPY₁₉₀ _{(SEQ ID NO: 9)} | | | |

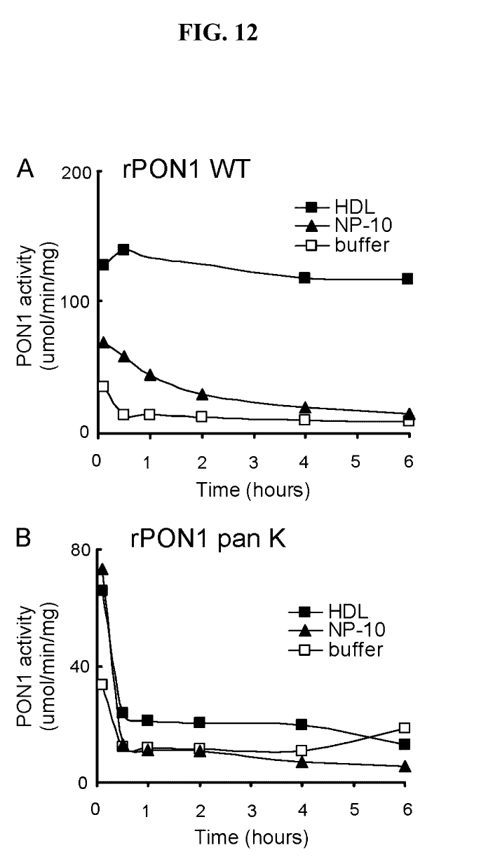

PON1 binding to HDL is known to stabilize PON1 catalytic activity at 37° C., with both protein (apoA-I) and lipid phase of HDL contributing to the overall docking and stabilization of PON1 on the HDL particle (refs. 29,69,70; herein incorporated by reference in their entireties). Experiments were conducted during development of embodiments herein to analyze the importance of PON1 Leu9, Tyr185 and Tyr293 in the ability of HDL to promote PON1 stabilization. To speed inactivation, rPON1 (WT versus pan K form) was incubated with a calcium chelator (EDTA) and a reducing agent, βmercaptoethanol, in the presence or absence of rHDL or detergent (0.1% tergitol (NP-10)) and the rate of inactivation of PON1 determined by monitoring arylesterase activity (FIG. 12A,B). This approach has been used to examine and characterize human and rabbit PON activity stabilities (ref 71,72; herein incorporated by reference in their entireties). A very slow inactivation of WT rPON1 was observed in the presence of rHDL, consistent with previous reports that HDL particles stabilize PON1 activity (72). Addition of detergent (0.1% tergitol, NP-10) to WT rPON1 also showed moderate resistance from inactivation of WT rPON1, while buffer alone showed relatively rapid decay in activity. In contrast, no protective effects were observed when the pan K rPON1 mutant was incubated with either rHDL or detergent (0.1% tergitol, NP-10), indicating PON1 Leu9, Tyr185 and Tyr293 additionally contribute to the HDL dependent stabilization of PON1 activity to thermal denaturation and detergents.

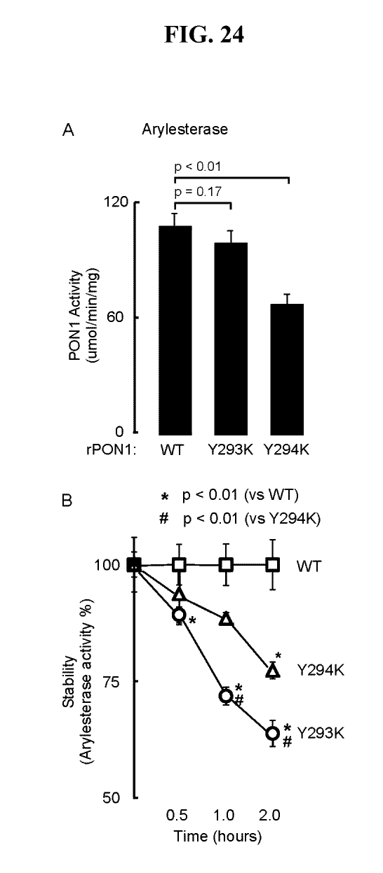

Since PON1 Tyr293 is dominantly photo-labeled by pac-PC compared with the adjacent Tyr294 residue, the functional significance of each residue to HDL binding/stabilization of PON1 activity was assessed. PON1 Tyr293 and Tyr294 were individually mutated to Lys, and their PON1 activity (measured as arylesterase activity) were determined. PON1 Y293K and Y294K both possess catalytic activity. Y294K begins with somewhat reduced activity compared to WT enzyme. To explore the strength and functional impact of the HDL-PON1 interaction with each PON1 residue, the stability of each mutant (Y293K vs Y294K) to thermal denaturation at 37° C. in the presence vs. absence of HDL was further examined, since studies show this is an excellent functional read out of PON1-HDL interaction. Incubation of either WT or both mutants in buffer alone at 37° C. (without HDL) resulted in rapid loss of activity. However, when HDL is present, WT PON1 is quite stable at 37° C., and the Y293K PON1 mutant shows the fastest rate of inactivation. Thus, while PON1 WT and Y293K mutant had almost the same activity and Y294K starts with less activity at T=0, upon incubation at 37° C. in the presence of HDL, the rate of Y293K loss in activity is faster than either Y294K or WT PON1, and comparison of Y293K vs. Y294K shows significant difference as well (FIG. 24). This result is consistent with residue Y293 playing a more important role in the PON1-HDL interaction relative to Y294.

All publications and patents provided herein incorporated by reference in their entireties. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising: (a) a lipid moiety, (b) an activatable moiety, and (c) a taggable moiety; wherein the activatable moiety and the taggable moiety are independently covalently linked to the lipid moiety.

2. The composition of claim 1, wherein the activatable moiety is a photoactivatable moiety.

3. The composition of claim 2, wherein the photoactivatable moiety comprises a functional group selected from the group consisting of: an aryl azide, an azido-methyl-coumarin, a benzophenone, an anthraquinone, a diazo compound, a diazirine ring, and a psoralen derivative.

4. The composition of claim 2, wherein the photoactivatable moiety comprises a diazirine ring.

5. The composition of claim 1, wherein the taggable moiety is a clickable moiety.

6. The composition of claim 5, wherein the clickable moiety comprises a function group selected from the group consisting of: an alkyne, an azide, a transcyclooctene, a tetrazine groups, and a dibenzocyclooctyne.

7. The composition of claim 6, wherein the clickable moiety comprises an alkyne.

8. The composition of claim 7, wherein the alkyne is a terminal alkyne.

9. The composition of claim 1, wherein the lipid moiety is selected from the group consisting of: a phospholipid, a glyceride, a sphingolipid, an eicosanoid, and a fatty acid.

10. The composition of claim 9, wherein the lipid moiety is a glyceride selected from a monoglyceride, diglyceride, and triglyceride.

11. The composition of claim 9, wherein the lipid moiety is a phospholipid.

12. The composition of claim 11, wherein the phospholipid is selected from phosphatidylethanolamine (PE), phosphatidylcholine (PC), phosphatidylserine (PS), phosphatidylglycerol (PG), phosphatidylinositol (PI), glycophosphatidylinositol (GPI), plasmalogens, cardiolipin, and sphingomyelin.

13. A system comprising:
a) a lipid-based probe comprising the composition of claim 1, and
b) a tagging element comprising a functional portion and a reactive portion,
wherein the reactive portion has the capacity to interact with the taggable moiety of the lipid-based probe to form a covalent bond or stable non-covalent interaction.

14. The system of claim 13, wherein the taggable moiety and reactive portion of the tagging agent are selected from: alkyne and azide groups, transcyclooctene and tetrazine groups, and dibenzocyclooctyne and azide groups.

15. A method of associating a functional portion of a tagging agent with a protein or protein complex within a lipid-containing structure, comprising:
(a) contacting the lipid-containing structure, or a system comprising the lipid-containing structure, with a lipid-based probe comprising the composition of claim 1, such that the lipid-based probe is incorporated into the lipid-containing structure;
(b) activating the activatable moiety to initiate formation of covalent bonding with the protein or protein complex; and
(c) contacting the taggable moiety of the lipid-based probe with the tagging agent, such that a reactive portion of the tagging agent forms a covalaent bond or stable non-covalent interaction with the taggable moiety, thereby associating the functional portion of the tagging agent with the protein or protein complex within the lipid-containing structure via the lipid-based probe.

16. The method of claim 15, further comprising a step of base-catalyzed hydrolysis of lipid side chains.

17. A method of identifying interactions between a protein or protein complex and lipids within a lipid-containing structure, comprising:
(a) contacting the lipid-containing structure, or a system comprising the lipid-containing structure, with a lipid-based probe comprising the composition of claim 1, such that the lipid-based probe is incorporated into the lipid-containing structure;
(b) activating the activatable moiety to initiate formation of covalent bonding with the protein or protein complex;
(c) contacting the taggable moiety of the lipid-based probe with the tagging agent, such that a reactive portion of the tagging agent forms a covalaent bond or stable non-covalent interaction with the taggable moiety;
(d) isolating the protein or protein complex using a functional portion of the tagging agent; and
(e) analyzing the protein or protein complex to identify residues bound to the lipid-based probe.

18. The method of claim 17, further comprising a step of base-catalyzed hydrolysis of lipid side chains.
